(19) United States
(12) Patent Application Publication
(10) Pub. No.: US 2018/0200405 A1
(43) Pub. Date: Jul. 19, 2018

(54) METHODS OF PREPARING ECM SCAFFOLDS AND HYDROGELS FROM COLON

(71) Applicant: University of Pittsburgh-Of the Commonwealth System Of Higher Education, Pittsburgh, PA (US)

(72) Inventors: Stephen F. Badylak, West Lafayette, IN (US); Timothy Joseph Keane, Jr., Wellsboro, PA (US)

(21) Appl. No.: 15/742,676

(22) PCT Filed: Jul. 8, 2016
(86) PCT No.: PCT/US2016/041489

§ 371 Date: Jan. 8, 2018

Related U.S. Application Data
(60) Provisional application No. 62/190,907, filed on Jul. 10, 2015.

(51) Int. Cl. A61L 27/36; A61L 27/52

(52) U.S. Cl. CPC A61L 27/3687; A61L 27/52; A61L 27/3629

(57) ABSTRACT

Provided herein is a method for producing decellularized colonic extracellular matrix material. A decellularized colonic extracellular matrix material also is provided, along with uses for the material. Methods of use of the decellularized colonic extracellular matrix material also are provided, including methods of treating a defective, diseased, or damaged tissue or organ in a patient, and/or methods of treating esophageal disease, short bowel syndrome, ulcerative colitis, Crohn's disease, or mucositis in a patient.

The present applications claims priority to U.S. Provisional Patent Application No. 62/190,907, filed Jul. 10, 2015, the content of which is incorporated herein by reference in its entirety.

Methods of preparing an ECM material from colon tissue are provided, along with methods of use of the tissue and related compositions

The gastrointestinal (GI) tract is composed of a series of hollow muscular tubes that perform a variety of functions including mastication, digestion, motility, nutrient absorption, and waste excretion, among others. Such functional diversity requires organization of specialized cell and tissue types, and repair following injury is imperative for the health of the host. Pathologies such inflammatory bowel disease affect up to 4 million patients per year and short bowel syndrome affects an additional 20,000 individuals in the United States alone. Diseases such as these have very limited therapeutic options and are the cause of tremendous morbidity and health care expenditures. Biomaterials and/or regenerative medicine strategies to address such problems will require the creation of a microenvironment that supports the cultivation, recruitment, differentiation, and maintenance of the specialized cell types required for normal GI function.

Biomaterial-mediated approaches to GI replacement must not only provide a mechanical support structure for cell growth but also be amenable to cell infiltration, allow for gas and nutrient exchange, and be compatible with the host innate immune system. Both synthetic and biologic scaffold materials have been manufactured and studied for GI repair/replacement applications and each are associated with their respective advantages and disadvantages (Bitar K N, et al. Intestinal tissue engineering: current concepts and future vision of regenerative medicine in the gut. Neurogastroenterol Motil. 2012; 24(1): 7-19 and Shin H, et al. Biomimetic materials for tissue engineering. Biomaterials. 2003; 24(24): 4353-64). Synthetic scaffolds such as poly-lactic acid or poly-caprolactone allow for tunable materials that can be tailored for specific applications. However, synthetic scaffolds invariably elicit pro-inflammatory and/or a foreign body response upon implantation that may result in encapsulation, fibrosis, and loss of function. Compatibility of scaffold materials with the host immune system has been shown to be a critical determinant of downstream functional tissue remodeling.

Biologic scaffold materials, such as those derived from decellularized extracellular matrix (ECM), can provide a compatible and instructive template for endogenous cell infiltration and differentiation, recapitulate the natural niche, and degrade to allow for complete host tissue replacement with associated release or exposure of bioactive matricryptic peptide sites. Implanted ECM bioscaffolds promote a favorable host immune response by induction of an M2-like macrophage phenotype. This immune modulation is typically associated with a functional constructive remodeling outcome. However, the properties and composition of ECM bioscaffolds are often variable and are critically dependent on factors such as source tissue anatomic site and age, use of chemical cross-linking agents, method of decellularization, manufacturing processes, and terminal sterilization methods, among others. The potential benefits of utilizing ECM scaffolds derived from homologous source tissue include the retention of tissue-specific cell phenotypes, enhancing tissue-specific differentiation, and promoting chemotaxis and proliferation of progenitor cells. Previous reports have shown that regions of the porcine GI system such as the small intestine (i.e., SIS) and esophagus can be decellularized and retain essential ultrastructural components, endogenous growth factors, and biomechanical strength. The suitability of these scaffolds for GI repair applications including treatment of esophageal disease, short bowel syndrome, ulcerative colitis, Crohn's disease, or mucositis, has not been extensively investigated.

### BRIEF DESCRIPTION OF THE DRAWINGS

FIGS. 1A-1D. Decellularization efficacy. (FIG. 1A) The presence of nuclei in the decellularized tissue was assessed by hemotoxylin and eosin (H&E) staining and 4′,6-diamidino-2-phenylindole (DAPI) staining. (FIG. 1B) DNA concentration was quantified using PicoGreen® assay. (FIG. 1C) The fragment length of residual DNA was visualized by gel electrophoresis. (FIG. 1D) Residual cell membrane components were quantified using EnzyChrom™ phospholipid assay. Scale bar=200 μm. **=p<0.01.

FIGS. 2A and 2B. Composition and ultrastructure. (FIG. 2A) The presence and distribution of laminin and fibronectin was assessed by immunohistochemical staining. (FIG. 2B)

The ultrastructure of the luminal and abluminal surfaces of the scaffold was visualized at low and high (inset) magnification. Scale bar in 2A=200 μm. Scale bar in 2B=50 μm.

FIGS. 3A-3E. Biochemical composition. The retention of biochemical constituents in coECM was compared to native colonic tissue. (FIG. 3A) The concentration of sulfated glycosaminoglycans (sGAGs) was measured using Blyscan™ assay. (FIG. 3B) Non-sulfated GAG hyaluronic acid (HA) content was measured using an ELISA. (FIG. 3C) Fibrilliar collagen was quantified using Sircol™ assay. The presence of two growth factors, bFGF (FIG. 3D) and VEGF (FIG. 3E), was detected using ELISA kits. *=p<0.05, **=p<0.01.

FIGS. 4A-4C. Scaffold mechanical properties. (FIG. 4A) The response of the scaffold to equibiaxial stress was assessed using planar biaxial testing. (FIG. 4B) Maximum strain of the scaffold at a stress of 250 kPa was quantified in the longitudinal and circumferential direction. (FIG. 4C) The suture retention strength of multi-laminate scaffolds was compared prior to in-vivo implantation. *=p<0.05, **=p<0.01.

FIGS. 5A-5F. Hydrogel turbidometric and rheological properties. (FIG. 5A) Two concentrations of coECM hydrogel, 4 and 8 mg/mL, were formed in a ring mold and compared macroscopically. Turbidometric anaylsis was used to measure the t_{lag} (FIG. 5B), t_{1/2} (FIG. 5C), and rate of gelation (FIG. 5D) of the hydrogel at two different concentrations. Parallel plate rheology was used to measure the viscosity of the pre-gel (FIG. 5E) and maximum storage modulus of the hydrogel (FIG. 5F). Scale bar=1 cm, *=p<0.05, **=p<0.01.

FIGS. 6A-6D. In-vitro cell response. (FIG. 6A) Intestinal epithelial cells cultured on coECM scaffold, coECM hydrogel, XL-coECM, and native submucosa were stained with LIVE/DEAD® cell viability dye and (FIG. 6B) the percentage of live and dead cells were quantified. (FIG. 6C) Bone marrow derived macrophages were cultured in the presence of enzymatically digested coECM and immunolabeled for F4/80 (pan macrophage), iNOS (M1), and Fizz1 (M2). Controls included MCSF (baseline), IFNγ+LPS (M1), IL-4 (M2), and pepsin (digestion buffer). (FIG. 6D) The percentage of cells expressing the markers indicative of M1 and M2 phenotypes was quantified and compared. **=p<0.01.

FIGS. 7A-7D. Host response. The host response to coECM scaffold and hydrogel was compared in-vivo to XL-coECM and native submucosa in a rat abdominal defect model. (FIG. 7A) Representative H&E images show the histologic response at 14 and 35 days. (FIG. 7B) The combined histologic score at each time point was quantified and compared across groups. (FIG. 7C) The macrophage response at 14 days post-surgery was analyzed by immunofluorescent staining for M2 indicator CD206 (green), M1 indicator CD86 (orange), and pan-macrophage CD68 (red). (FIG. 7D) The ratio of M2 (CD68+/CD206+) to M1 (CD68±/CD86+) was quantified and compared across groups. Dashed lines indicate interface between scaffold and underlying muscle. *=p<0.05, ** =p<0.01.

FIG. 8. Storage Modulus of various hydrogels from varying source tissues. Comparison of rigidity of 8 mg/ml hydrogels from varying source tissues.

FIG. 9. Comparison of different decellularization protocols. Various protocols for decellularization were performed and DNA concentration was quantified using PicoGreen® assay.

FIG. 10. Comparison of different decellularization protocols. Comparison of deoxycholate and triton decellularization protocols. Remaining DNA concentration was quantified using PicoGreen® assay.

FIG. 11. Comparison of different decellularization protocols. Comparison of deoxycholate and deoxycholate+triton decellularization protocols. Remaining DNA concentration was quantified using PicoGreen® assay.

FIG. 12. Measurement of DNA concentration. Decellularization protocol using a ratio of 10 ml of Trypsin/EDTA to 1 g of ECM and a ratio of 19 ml 4% deoxycholate to 1 g of ECM yielded in around 50 ng/ml remaining DNA content, which is indicative of effective decellularization. The four bars indicate four separate preparations of coECM following the deoxycholate procedure

FIG. 13 is a photograph of an SDS PAGE gel showing distinct banding patterns of pepsin-digested ECM from various tissue sources.

FIGS. 14A and 14B are a graph and fluorescent photomicrographs, respectively showing colon ECM supports macrophage activation towards M2.

FIGS. 15A-15D are Western blots (FIGS. 15A and 15C) and graphs (FIGS. 15B and 15D) showing iNOS and CD0206 markers according to Example 9.

FIG. 16 is a graph showing the metabolism of macrophages in contact with the indicated materials according to Example 10.

FIG. 17 is a graph showing antimicrobial activity of the indicated materials according to Example 11.

FIG. 18 provides photomicrographs showing SIS induces the expression of the antimicrobial peptide cathelicidin LL-37.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more.

As used herein, the term “patient” or “subject” refers to members of the animal kingdom including but not limited to human beings and “mammal” refers to all mammals, including, but not limited to human beings.

As used herein, the “treatment” or “treating” of a wound or defect means administration to a patient by any suitable dosage regimen, procedure and/or administration route of a composition, device or structure with the object of achieving a desirable clinical/medical end-point, including attracting progenitor cells, healing a wound, correcting a defect, etc.

As used herein, the terms “comprising,” “comprise” or “comprised,” and variations thereof, are open ended and do not exclude the presence of other elements not identified. In contrast, the term “consisting of” and variations thereof is intended to be closed, and excludes additional elements in anything but trace amounts.

As used herein, the terms “extracellular matrix” and “ECM” refer to a natural scaffolding for cell growth obtained from the decellularization or devitalization of tissue. Extracellular matrix (ECM) is a complex mixture of structural and non-structural biomolecules, including, but not limited to, collagens, elastins, laminins, glycosaminoglycans, proteoglycans, antimicrobials, chemoattractants, cytokines, and growth factors. In mammals, ECM often comprises about 90% collagen, in its various forms. The composition and structure of ECM varies depending on the source of the tissue. For example, small intestine submucosa (SIS), urinary bladder matrix (UBM), liver stroma ECM, and dermal ECM each differ in their overall structure and composition due to the unique cellular niche needed for each tissue.

As used herein, the terms “intact extracellular matrix” and “intact ECM” refers to an extracellular matrix that retains activity of at least a portion of its structural and non-structural biomolecules, including, but not limited to, collagens, elastins, laminins, glycosaminoglycans, proteoglycans, antimicrobials, chemoattractants, cytokines, and/or growth factors, such as, without limitation comminuted ECM as described herein. The activity of the biomolecules within the ECM can be removed chemically or mechanically, for example, by cross-linking and/or by dialyzing the ECM. Intact ECM has not been subjected to a dialysis and/or a cross-linking process prior to solubilization in an acid protease as described herein.

ECM, for example intact ECM, is typically prepared by the decellularization and/or devitalization of tissues. As indicated above, decellularization may be performed to prevent a pro-inflammatory response. As such, a decellularized or devitalized ECM product or a decellularized or devitalized intact ECM product preferably is used herein to refer to ECM material that is decellularized to the extent that a pro-inflammatory response, and thus growth of fibrotic tissue is not is not elicited to any substantial degree in favor of constructive remodeling.

By “biocompatible”, it is meant that a device, scaffold composition, etc. is essentially, practically (for its intended use) and/or substantially non-toxic, non-injurious or non-inhibiting or non-inhibitory to cells, tissues, organs, and/or organ systems that would come into contact with the device, scaffold, composition, etc.

In general, the method of preparing an ECM-derived gel requires the isolation of ECM from an animal of interest and from a tissue or organ of interest, for example vertebrate or mammalian, such as, without limitation, human, monkey, pig, cattle, or sheep colon tissue. “Colon” refers to the ascending, transverse, descending, and sigmoid colon, as well as the rectum and anal canal. In one aspect, the ECM includes the basement membrane portion of the ECM. In another aspect, the ECM does not include the basement membrane portion of the ECM. In certain aspects, the ECM includes at least a portion of the basement membrane.

As used herein, the term “derive” and any other word forms or cognates thereof, such as, without limitation, “derived” and “derives”, refers to a component or components obtained from any stated source by any useful method. For example and without limitation, an ECM-derived gel refers to a gel comprised of components of ECM obtained from any tissue by any number of methods known in the art for isolating ECM. In another example, mammalian tissue-derived ECM refers to ECM comprised of components of a particular mammalian tissue obtained from a mammal by any useful method.

According to one aspect, a method for producing decellularized colonic extracellular matrix material is provided. The decellularized colonic extracellular matrix material is provided as a device, such as a sheet or a tube, or as a pre-gel or a gel. When provided as a gel, the gel is reverse gelling, or can be said to exhibit reverse thermal gelation, in that it forms a gel (sol to gel transition) upon an increase in temperature. The lower critical solution temperature (LCST) in a reverse gel is a temperature below which a reverse-gelling polymer is soluble in its solvent (e.g. water or an aqueous solvent). As the temperature rises above the LCST in a reverse gel, a hydrogel is formed. The general concept of reverse gelation of polymers and its relation to LCST are broadly known in the chemical arts. The materials described herein are prepared, for example, from decellularized or devitalized, intact ECM as described below. A gel is prepared by digestion of ECM material with an acid protease, neutralization of the material to form a pre-gel, and then raising or maintaining the temperature of the pre-gel above the LCST of the pre-gel to cause the pre-gel to gel. As used herein, the term “gel” includes hydrogels. The transition temperature for acid-protease-digested ECM materials from solution to gel is typically within the range of from 10° C. to 40° C. and any increments or ranges therebetween, for example from 20° C. to 35° C. For example, the pre-gel can be warmed to 37° C. to form a hydrogel.

Tissue for preparation of ECM and ECM-derived pre-gel solutions and gels can be harvested in a large variety of ways, and once harvested, a variety of portions of the harvested tissue may be used. For example and without limitation, in one embodiment, the ECM is isolated from harvested porcine colon. Excess connective tissue and residual waste are removed from the colon, and the tissue is optionally frozen. Colonic submucosa is obtained by any suitable method, for example by manually isolated from the surrounding tissue.

In the method, the colonic tissue, such as the colonic submucosa, is first delipidized using a combination of chloroform and lower alcohols, such as a C₁-C₄ alcohol, including methanol, ethanol, n-propanol, isopropanol, n-butanol, isobutanol, 2-butanol or mixtures thereof. The tissue is incubated in chloroform, or a mixture of chloroform and a C₁-C₄ alcohol, such as chloroform:methanol, and, in one aspect, is then rinsed in a lower alcohol, e.g., a C₁-C₄ alcohol, such as ethanol. The delipidized tissue is then washed and incubated with a protease, such as trypsin, and then subsequently washed with a deoxycholic acid or salt thereof, such as sodium deoxycholate. Alternative proteases to trypsin include, for example, other proteases active at a neutral or near-neutral pH, such as, without limitation: chymotrypsin, thermolysin, pepsin, Arg-C, Lys-N, Asp-N, and Lys-C. The tissue is then disinfected, e.g. with a peroxyacid, such as peracetic acid, thereby producing an ECM composition. In one non-limiting embodiment, the colon tissue is not crosslinked or dialyzed at any step, such that the ECM material is an intact ECM material.

In one aspect, the decellularized colonic extracellular matrix material is then dried, either lyophilized (freeze-dried) or air dried. The ECM material is optionally comminuted at some point, for example prior to enzymatic digestion in preparation of a gel, for example prior to or after drying. Dried ECM material produced by the methods above, when comminuted is comminuted by any effective method, including, but not limited to, tearing, milling, cutting, grinding, and shearing. The comminuted ECM can also be further processed into a powdered form by methods, for example and without limitation, such as grinding or milling in a frozen or freeze-dried state. As used herein, the term “comminute” and any other word forms or cognates thereof, such as, without limitation, “comminution” and “comminuting”, refers to the process of reducing larger particles into smaller particles, including, without limitation, by grinding, blending, shredding, slicing, milling, cutting, shredding. ECM can be comminuted while in any form, including, but not limited to, hydrated forms, frozen, air-dried, lyophilized, powdered, sheet-form.

As indicated above, in one aspect, the decellularized colonic extracellular matrix material is solubilized to provide a pre-gel or a gel. In order to prepare solubilized material, ECM, for example comminuted ECM, is digested with an acid protease in an acidic solution to form a digest solution. As used herein, the term “acid protease” refers to an enzyme that cleaves peptide bonds, wherein the enzyme has increased activity of cleaving peptide bonds in an acidic pH. For example and without limitation, acid proteases include pepsin and trypsin and mixtures thereof.

As an example, a digest solution of decellularized colonic extracellular matrix material and an acid protease is kept at a constant stir for a certain amount of time at room temperature. The digest solution can be used immediately or be stored at -20° C. or frozen at, for example and without limitation, −20° C. or −80° C. In certain aspects, the ECM digest is snap frozen in liquid nitrogen. To form a “pre-gel” solution, the pH of the digest solution is raised to a pH between 7.2 and 7.8. The pH can be raised by any useful method, and in one aspect, the pH is raised by adding one or more of a base, or an isotonic buffered solution, for example and without limitation, NaOH or PBS at pH 7.4. The method optionally does not include a dialysis step prior to gelation, yielding a more-complete ECM-like matrix that typically gels at 37° C. more slowly than comparable collagen or dialyzed ECM preparations. In certain embodiments, dialysis, or similar methods, are not used. The gel therefore retains more of the qualities of native ECM due to retention of many native soluble factors, such as, without limitation, cytokines. These factors contribute to chemoattraction of cells and proper rearrangement of tissue at the site of injury, rather than fibrous response that leads to unwanted scarring. In other aspects, the ECM is dialyzed prior to gelation to remove certain soluble components.

As used herein, the term “isotonic buffered solution” refers to a solution that is buffered to a pH between 7.2 and 7.8, e.g., pH 7.4, and that has a balanced concentration of salts to promote an isotonic environment. As used herein, the term “base” refers to any compound or a solution of a compound with a pH greater than 7. For example and without limitation, the base is an alkaline hydroxide or an aqueous solution of an alkaline hydroxide. In certain embodiments, the base is

NaOH or NaOH in PBS. This “pre-gel” solution can, at that point be incubated at a suitably warm temperature, for example and without limitation, at about 37° C. to gel.

In the method of preparing an ECM gel, the ECM may be partially or completely digested with the acid protease, such as pepsin. The digested ECM is then neutralized to a pH of 7.2-7.8, e.g., 7.3-7.5 or 7.4 and the neutralized and digested ECM material is gelled at a temperature above its

Lower Critical Solution Temperature. For example, in one aspect, the decellularized ECM material is digested less completely than a digestion of 1 mg/mL lyophilized, powdered ECM material with 1 mg/mL pepsin in 0.01 M HCl for 48 hours. Alternately, in another embodiment, the decellularized ECM material is digested less completely than a digestion of 10 mg/mL lyophilized, powdered ECM material with 1 mg/mL pepsin in 0.01 M HCl for 48 hours. This degree of digestion can be determined by comparison on a gel, or by ascertaining the degree of degradation of hyaluronic acid, for example by Western blot (anti-hyaluronic acid antibodies are commercially-available from multiple sources) or chromatographic methods, as are broadly known. For example in one aspect, hyaluronic acid is digested less than 50%, 40%, 30%, 25%, 20% or 10% and/or sulfated glycosaminoglycans are digested less than 50%, 40%, 30%, 25%, 20% or 10%. In one aspect, at least 85%, 90, or 95% of phospholipids are removed from the colon tissue, as compared to the original tissue. In another aspect, at least 95% of the DNA of the colon tissue is removed, as compared to the original colon tissue. In another aspect, any residual DNA in the colon tissue (that is DNA remaining in the material after processing as described) is in fragments of <200 bases in length. In yet another aspect, the amount of collagen in the colon tissue is enriched by at least 3 fold (that is, the percentage by weight of collagen in the material is increased by at least a factor of three) by the methods of making a decellularized colonic extracellular matrix material as described herein.

In use, the decellularized colonic extracellular matrix material are sheets and are applied, e.g., sutured or glued into place, for example onto, or integral with colon tissue of a patient, such as in repair of a traumatic injury, defect, or surgical resection. The ECM gel can be injected, sprayed, painted, poured, or otherwise applied to a surface of a tissue, e.g., the colon of a patient. Depending on the final use of the product, the composition may be applied or administered in a variety of ways, either as a dry, e.g., sheets or as a lyophilized powder, or as a solution, gel or pre-gel. The composition can be administered by itself, or with a device or composition. For example, the composition can be absorbed into, adsorbed onto, mixed into or otherwise co-administered with a cell-growth scaffold, such as an isotropic or anisotropic mass of fibers of synthetic and/or natural polymer(s), such as an electrodeposited, wet or dry spun, 3D printed, molded, or otherwise formed polymeric structure prepared from biocompatible polymeric materials, as are broadly known in the regenerative medical field, such as collagen, polyester, polyurethane, poly(ester urethane) urea, and poly(ether ester urethane) urea copolymers, and other suitable polymeric materials, such as are disclosed, for example and without limitation in U.S. Pat. Nos. 8,535,719; 8,673,295; 8,889,791; 8,974,542 and 9,023,972. The compositions described herein also can be mixed into polymeric compositions prior to or along with deposition of polymeric fibers or formation of structures. Alternatively, where the colon ECM product is not formed into a gel, ECM gel and/or synthetic polymers may be absorbed into, adsorbed onto or otherwise combined with the ECM product. In one aspect, a decellularized colonic extracellular matrix material as described herein is applied to and delivered from an ECM material, such as any commercial ECM material, such as those described above.

Likewise, the decellularized colonic extracellular matrix material described herein can be applied to or incorporated into, by any suitable method, a non-woven material, such as a bandage, a suture, an implant, such as a ceramic, metal, or polymeric implant, for example a prosthesis, artificial or otherwise-modified vessel, a valve, an intraocular lens, a tissue transplant or implant.

As used herein, the term “coat”, and related cognates such as “coated” and “coating,” refers to a process comprising of covering an inorganic structure with a composition described herein. For example and without limitation, coating of an inorganic structure with ECM-derived gel can include methods such as pouring, embedding, layering, dipping, spraying. Ultrasonication may be used to aid in coating of an inorganic structure with the ECM-derived gel. As used herein, the term “ultrasonication” refers to the process of exposing ultrasonic waves with a frequency higher than 15 kHz and lower than 400 kHz.

In another aspect, the decellularized colonic extracellular matrix material in the form of a pre-gel is coated onto a biocompatible structural material, such as a metal, an inorganic calcium compound such as calcium hydroxide, calcium phosphate or calcium carbonate, or a ceramic composition. Non-limiting examples of suitable metals are cobalt-chrome alloys, stainless steel alloys, titanium alloys, tantalum alloys, titanium-tantalum alloys, which can include both non-metallic and metallic components, such as molybdenum, tantalum, niobium, zirconium, iron, manganese, chromium, cobalt, nickel aluminum and lanthanum, including without limitation, CP Ti (commercially pure titanium) of various grades or Ti 6Al 4V (90% wt. Ti, 6% wt. Al and 4% wt. V), stainless steel 316, Nitinol (Nickel-titanium alloy), titanium alloys coated with hydroxyapatite. Metals are useful due to high strength, flexibility, and biocompatibility. Metals also can be formed into complex shapes and many can withstand corrosion in the biological environments, reduce wear, and not cause damage to tissues. In one non-limiting example, the metal is femoral or acetabular component used for hip repair. In another example, the metal is a fiber or other protuberance used in permanent attachment of a prosthesis to a patient. Other compositions, including ceramics, calcium compounds, such as, without limitation, aragonite, may be preferred, for example and without limitation, in repair of or re-shaping of skeletal or dental structures. Combinations of metal, ceramics and/or other materials also may prove useful. For instance, a metal femoral component of a hip replacement may comprise a ceramic ball and/or may comprise a plastic coating on the ball surface, as might an acetabular component.

In certain aspects, the composition is used for release of one or more therapeutic agents within a patient's body and/or incorporates one or more therapeutic agents. For example, at least one therapeutic agent is added to the composition described herein before it is implanted in the patient or otherwise administered to the patient. Generally, the therapeutic agents include any substance that can be coated on, embedded into, absorbed into, adsorbed to, or otherwise attached to or incorporated onto or into the composition described herein or incorporated into a drug product that would provide a therapeutic benefit to a patient. Non-limiting examples of such therapeutic agents include: growth factors, chemoattractants, cytokines, antimicrobial agents, emollients, retinoids, and topical steroids. Each therapeutic agent may be used alone or in combination with other therapeutic agents. For example and without limitation, a composition comprising neurotrophic agents or cells that express neurotrophic agents may be applied to a wound that is near a critical region of the central nervous system, such as the spine.

In certain aspects, the therapeutic agent is a growth factor, such as a neurotrophic or angiogenic factor, which optionally may be prepared using recombinant techniques. Non-limiting examples of growth factors include basic fibroblast growth factor (bFGF), acidic fibroblast growth factor (aFGF), vascular endothelial growth factor (VEGF), hepatocyte growth factor (HGF), insulin-like growth factors 1 and 2 (IGF-1 and IGF-2), platelet derived growth factor (PDGF), stromal derived factor 1 alpha (SDF-1 alpha), nerve growth factor (NGF), ciliary neurotrophic factor (CNTF), neurotrophin-3, neurotrophin-4, neurotrophin-5, pleiotrophin protein (neurite growth-promoting factor

I), midline protein (neurite growth-promoting factor 2), brain-derived neurotrophic factor (BDNF), tumor angiogenesis factor (TAF),corticotrophin releasing factor (CRF), transforming growth factors a and β (TGF-α and TGF-β), interleukin-8 (IL-8), granulocyte-macrophage colony stimulating factor (GM-CSF), interleukins, and interferons. Commercial preparations of various growth factors, including neurotrophic and angiogenic factors, are available from R & D Systems, Minneapolis, Minn.; Biovision, Inc, Mountain View, Calif.; ProSpec-Tany TechnoGene Ltd., Rehovot, Israel; and Cell Sciences®, Canton, Mass.

In certain aspects, the therapeutic agent is an antimicrobial agent, such as, without limitation, isoniazid, ethambutol, pyrazinamide, streptomycin, clofazimine, rifabutin, fluoroquinolones, ofloxacin, sparfloxacin, rifampin, azithromycin, clarithromycin, dapsone, tetracycline, erythromycin, ciprofloxacin, doxycycline, ampicillin, amphotericin B, ketoconazole, fluconazole, pyrimethamine, sulfadiazine, clindamycin, lincomycin, pentamidine, atovaquone, paromomycin, diclazuril, acyclovir, trifluorouridine, foscarnet, penicillin, gentamicin, ganciclovir, itraconazole, miconazole, Zn-pyrithione, and silver salts such as chloride, bromide, iodide and periodate.

In certain aspects, the therapeutic agent is an anti-inflammatory agent, such as, without limitation, an NSAID, such as salicylic acid, indomethacin, sodium indomethacin trihydrate, salicylamide, naproxen, colchicine, fenoprofen, sulindac, diflunisal, diclofenac, indoprofen, sodium salicylamide; an anti-inflammatory cytokine; an anti-inflammatory protein; a steroidal anti-inflammatory agent; or an anti-clotting agents, such as heparin. Other drugs that may promote wound healing and/or tissue regeneration may also be included.

In certain aspects, cells are added to the decellularized colonic extracellular matrix material. Non-limiting examples of useful cells include: stem cells, progenitor cells and differentiated cells; recombinant cells; muscle cells and precursors thereof; nerve cells and precursors thereof; mesenchymal progenitor or stem cells; bone cells or precursors thereof, such as osteoprogenitor cells.

Any useful cytokine, chemoattractant, drug or cells can be mixed into, mixed with, co-applied or otherwise combined with any decellularized colonic extracellular matrix material as described herein. For example and without limitation, useful components include growth factors, interferons, interleukins, chemokines, monokines, hormones, angiogenic factors, drugs and antibiotics. Cells can be mixed into the decellularized colonic extracellular matrix material or can be included on or within a sheet, tube or other device, such as a biological scaffold, combined with the decellularized colonic extracellular matrix material. In either case, when the substrate is seeded with cells, the cells can be grown and/or adapted to the niche created by incubation in a suitable medium in a bioreactor or incubator for a suitable time period to optimally/favorably prepare the composition for implantation in a patient. The substrate can be seeded with cells to facilitate in-growth, differentiation and/or adaptation of the cells. For example and without limitation, the cells can be autologous or allogeneic with respect to the patient to receive the composition/device comprising the gel. The cells can be stem cells or other progenitor cells, or differentiated cells. In one example, a layer of dermis obtained from the patient is seeded on a mold, for use in repairing damaged skin and/or underlying tissue.

As used herein, the terms “therapeutic agent” and “therapeutic agents” refer to any composition(s), such as drug(s) or active agent(s) having a preventative or therapeutic effect, including and without limitation, antibiotics, peptides, hormones, organic molecules, vitamins, supplements, factors, proteins and chemoattractants.

As used herein, the terms “cell” and “cells” refer to any types of cells from any animal, such as, without limitation, rat, mice, monkey, and human. For example and without limitation, cells can be progenitor cells, such as stem cells, or differentiated cells, such as endothelial cells, smooth muscle cells. In certain embodiments, cells for medical procedures can be obtained from the patient for autologous procedures or from other donors for allogeneic procedures.

In a further aspect, a commercial kit is provided comprising a composition described herein. A kit comprises suitable packaging material and the composition. In one non-limiting embodiment, the kit comprises a liquid, gelled or dried decellularized colonic extracellular matrix material as described herein in a vessel or container, which may be the packaging, or which may be contained within packaging. The vessel may be a vial, syringe, tube or any other container suitable for storage and transfer in commercial distribution routes of the kit. Likewise, a product, such as a device, gel, scaffolding, suture, prosthetic, mesh, etc. including one or both of the soluble or structural compositions described herein may be packaged appropriately for commercial distribution.

In another aspect, a method or treating a defective, diseased, or damaged tissue or organ in a patient comprising implanting, injecting or otherwise introducing the decellularized colonic extracellular matrix material in a patient is provided. In yet another aspect, a method of treating esophageal disease, short bowel syndrome, ulcerative colitis, Crohn's disease, or mucositis in a patient. The method comprises implanting, injecting or otherwise introducing the decellularized colonic extracellular matrix material according to any aspect provided herein, on or about defective, diseased, or damaged tissue or organ of the patient, such as to replace resected gastrointestinal tissue, such as esophageal, small intestine or colon tissue, e.g., colon tissue, in a patient.

### EXAMPLE 1

The objective of the present study was to prepare, characterize, and determine the in vitro and in vivo cytocompatibility of ECM bioscaffolds derived from porcine colon. DNA content, retention of ultrastructural and biochemical molecules, biomechanical properties, in vitro cytocompatibility, and the in vivo host macrophage response were examined both quantitatively and qualitatively and compared across sheet, hydrogel, cross-linked, and incompletely decellularized forms of porcine colon ECM.

Gastrointestinal pathologies, injuries, and defects affect millions of individuals each year. While there are diverse treatment options for these individuals, no ideal solution exists. The repair or replacement of gastrointestinal tissue therefore represents a large unmet clinical need. Biomaterials derived from extracellular matrix scaffolds have been effectively used to repair or replace numerous tissues throughout the body in both pre-clinical and clinical studies. Such scaffolds are prepared from decellularized tissues and the biochemical, structural, and biologic properties vary depending upon the source tissue from which the ECM is derived. The objective of the present study was to prepare, characterize, and determine the in vitro and in vivo cell response to ECM derived from porcine colon. Results of the present study show that porcine colon can be effectively decellularized while retaining biochemical and structural constituents of the source tissue. Two forms of coECM, scaffold and hydrogel, were shown to be cell-friendly and facilitate the polarization of macrophages towards an M2 phenotype both in-vitro and in-vivo.

### Materials and Methods

Preparation of Colonic ECM (coECM)

Colons were collected from market weight pigs (approximately 6 months of age and 260 lbs) at a local abattoir (Thoma's Meat Market, Saxonburg, Pa.). The colon was rinsed in water to remove contents and frozen at −20° C. until use. Colonic submucosa was mechanically isolated from the surrounding tissue and then delipidized and decellularized. Native colonic submucosa prior to delipidization and decellularization was used as a control group. For preparation of coECM, submucosa was subject to agitated washes of 2:1 (v/v) chloroform to methanol (30 min with stirring), 3 washes each of 100%, 90%, and 70% ethanol (5 minutes each), 3 washes of deionized water (5 min), 0.02% Trypsin/0.05% EDTA at a ratio of 10 ml of Trypsin/EDTA solution to 1 g of submucosa (1 h at 37° C.), twice with deionized water (5 min) 4% sodium deoxycholate at a ratio of 19 ml of 4% sodium deoxycholate to 1 g of submucosa (30 min), twice with deionized water (5 min), 4% sodium deoxycholate at a ratio of 19 ml of 4% sodium deoxycholate to 1 g of submucosa (30 min), deionized water (2×5 min), 0.1% peracetic acid (e.g., a ratio of 20 ml of peracetic acid to 1 gram of ECM) in 4% ethanol (2 h), PBS (15 min), deionized water (2×15min), and PBS (15 min). Solutions were agitated on a shaker at 300 rpm and room temperature unless otherwise stated.

A subset of coECM scaffolds was subjected to chemical cross-linking (XL) using 10 mM carbodiimide for 24 h at room temperature with constant stirring. The XL-coECM was then washed extensively in PBS for 4811 with stirring and then lyophilized to dry. For in-vitro cell growth, in-vivo implants, and suture retention strength experiments, the colonic submucosa, XL-coECM, and coECM were vacuum pressed to form a 4-layer device. The devices used for cell culture and in-vivo implantation were sterilized by ethylene oxide.

### Hydrogel Preparation

Hydrogels were prepared from coECM. Briefly, lyophilized scaffolds were powdered using a Wiley Mill and filtered through a 60 mesh screen (<250 μm particle size). The comminuted ECM was then digested in 1 mg/mL porcine pepsin (Sigma Aldrich, St. Louis, Mo.) in 0.01 N HCl for 48 h under constant stir rate at room temperature. Gelation was induced by neutralization at 4° C. of pH and salt concentration with the respective addition of one-tenth digest volume of 0.1 N NaOH and one-ninth digest volume of 10× PBS. Gelation was then achieved by placing the neutralized digest in a non-humidified incubator at 37° C. for 1 h for in-vitro studies. Gelation of coECM hydrogels for in-vivo studies was accomplished by direct injection of the neutralized digest over the site of abdominal wall defect. ECM concentrations of 4 and 8 mg/mL were prepared by turbidometric and rheologic assays. Cell culture and in-vivo experiments were conducted with 8 mg/mL coECM hydrogels.

### Determining Decellularization Efficacy

### Histologic Analysis

Scaffolds (native colonic submucosa and coECM) and native colon tissue were fixed in 10% neutral buffered formalin for 24 h. The fixed samples were then paraffin embedded and 5 μm sections were cut onto slides. Slides were stained with hematoxylin and eosin (H&E) or 4′,6-onDiamidino-2-phenylindole (DAPI) to visualize the presence of nuclear material.

### DNA Concentration and Fragment Length Analysis

Residual DNA content of the ECM was quantified by powdering samples with a Wiley Mill using a 60-mesh from separate preparations (n=4) of lyophilized coECM. Samples (100 mg) were digested in 0.1 mg/mL proteinase K digestion buffer at 50° C. for 24 hours. DNA was extracted twice in phenol/chloroform/isoamyl alcohol and centrifuged at 10,000 g (10 min at 4° C.). The aqueous phase, containing the DNA, was then mixed with 3 M sodium acetate and 100% ethanol, frozen on dry ice for 20 minutes, centrifuged at 10,000g (10 min at 4° C.), pouring off the supernatant, adding 70% ethanol, repeating centrifugation, removing supernatant, and drying the remaining DNA pellet. When dry, the pellet was resuspended in TE buffer (10mM Tris/1 mM EDTA) and the DNA concentration was quantified utilizing a PicoGreen Assay (Invitrogen) following manufacturer's instructions. The fragment length of remnant DNA in the samples was then visualized with gel electrophoresis on a 1% agarose gel with a 100 bp ladder (Invitrogen) containing ethidium bromide.

### Phospholipid Measurement

Homogenates were prepared from 40 mg of lyophilized and comminuted tissue or ECM in 2 mL of homogenization buffer (50 mM Iris pH 7.4/20 mM CaCl₂/0.5% Triton X-100). Samples were homogenized on ice 5 times for 15 sec using a PowerGen 500 Homegenizer (Fisher Scientific, Pittsburgh, Pa.). Samples were centrifuged at 2,000×g for 20 min at 4° C. and the supernatant extract was collected. A second extraction was completed on the remaining pellet, as above, using 1 mL of extraction buffer. The extracts were combined and measured for phospholipid content using EnzyChrom Phospholipid Assay Kit (BioAssay Systems, Hayward, Calif.) according to manufacturers instructions.

### Glycosaminoglycan and Growth Factor Measurement

The concentration of sulfated glycosaminoglycan (sGAG) and non-sulfated GAG in coECM samples was determined using the Blyscan Sulfated Glycosaminoglycan Assay Kit (Biocolor Ltd, Belfast, Northern Ireland) and Hyaluronan Quantikine ELISA Kit (R&D Systems, Minneapolis, MN), respectively. The concentration of non-sulfated GAG, hyaluranic acid (HA), was measured using neutralized pepsin digests as described above. Digested samples were assayed following the manufacturer's protocol, and the assay was performed in duplicate on three different coECM samples.

The concentration of basic fibroblast growth factor (bFGF) and vascular endothelial growth factor (VEGF) in urea-heparin extracts of coECM samples was determined with the Quantikine Human FGF basic Immunoassay, Human VEGF Immunoassay (R&D Systems). Each assay for bFGF and VEGF was performed in quadruplicate. The ELISA assays are cross-reactive with porcine growth factors and do not measure activity.

### Immunohistochemistry

Antigen retrieval was performed on de-paraffinized slides with 5 μm sections using a 0.01 M citrate buffer (pH=6) heated to 95-100° C. Slides were placed in the hot buffer for 20 min and subsequently rinsed in PBS (3×5 min). Sections were placed in pepsin solution (0.05% pepsin/0.01 M HCl) at 37° C. for 15 minutes. After rinsing in PBS (3×5 min), the samples were blocked in blocking buffer (2% goat serum/ i% bovine serum albumin/0.1% Triton X-100/0.1% Tween) for 1 hr at room temperature. The sections were then incubated in the blocking buffer with rabbit polyclonal laminin antibody (1:200 dilution, Abeam), or mouse monoclonal fibronectin (1:200 dilution, Abeam) overnight at 4° C. in a humidified chamber. Sections were subsequently rinsed in PBS (3×5 min). Endogenous peroxidase activity was quenched by rinsing sections in a 3% hydrogen peroxide in methanol solution for 30 min followed by rinsing in PBS (3×5 min). Biotinylated goat anti-rabbit or goat anti-mouse secondary antibodies (Vector Laboratories, Burlingame, Calif.) were diluted 1:200 in blocking buffer and added to the sections for 30 min at 25° C. and sections were subsequently rinsed in PBS (3×5 min). The slides were then incubated in detection solution (VectaStain® Elite ABC Reagent, Vector Laboratories) for 30 minutes at 37° C. After rinsing the slides, peroxidase substrate, 3,3′-diaminobenzadine (ImmPACT™ DAB, Vector Laboratories) was prepared per manufacturer instructions and slides were incubated while being visualized under a microscope to time the color change for subsequent section staining. Tissues were rinsed in water (3×5 min). Sections were dipped in hematoxylin (Thermo Shandon, Pittsburgh, Pa.) for 1 min for a nuclear counterstain and subsequently rinsed in PBS (3×5 min).

### Scanning Electron Microscopy

Scanning electron microscopy was used to examine the surface topology of the luminal and abluminal sides of native porcine colonic tissue, submucosal tissue, and colonic ECM. Samples were fixed in 2.5% glutaraldehyde in 1× PBS for 60 min, cut into blocks of 8 mm³, and washed thoroughly in 1× PBS three times at 15 min each. Samples were then fixed in 1% OsO4 in 1× PBS for 15 min each, dehydrated in graded series of alcohol (30-100%) baths for 15 min each. Samples were then critically point dried with hexamethyldisiloxane, mounted on studs, sputter coated and stored in a desiccator until imaged. SEM images were captured using a JEOL 6335F Field Emission SEM instrument with a backscatter detector.

Mechanical Testing of coECM Scaffolds

### Planar Biaxial Testing

Planar biaxial mechanical testing was performed as previously described (Wolf M T, et al. Polypropylene surgical mesh coated with extracellular matrix mitigates the host foreign body response. J Biomed Mater Res A. 2013). Briefly, a 15 mm×15 mm sample of each tested material was acquired. Thickness was measured from the center of each material using a Sterret® caliper model 1010. Four fiducial markers were placed in the center of the square on the luminal surface after the removal of excess loose connective tissue and fat. Deformations were measured optically by tracking this four marker array. Two loops of suture of equal length were attached to each side of the specimens with four stainless steel hooks, and 500 g Model 31 load cells (Honeywell) were used to acquire load values. Biaxial testing was conducted with the circumferential and longitudinal specimen axes aligned with the device axis and submerged in a bath at room temperature. The biaxial testing system was automated, allowing the marker locations and axial forces to be continuously recorded with custom marker tracking and data acquisition software (Billiar K L, et al. Biaxial mechanical properties of the natural and glutaraldehyde treated aortic valve cusp—Part I: Experimental results. J Biomech Eng. 2000; 122(1): 23-30).

Specimens were first preconditioned by cyclically loading the specimens to the desired maximum equibiaxial stress of 250 kPA for ten cycles using a cycle time of 30 s per cycle to quantify the quasi-static response. Immediately following the preconditioning cycles, the specimen was completely unloaded and imaged in its post-preconditioned free-floating configuration. The stress-stretch plot reported in this study start from a 5 g preload that is referenced to the post-precondition free float state, which was used to ensure test response repeatability. The response of the eight devices from each group was averaged after a three point linear interpolation at representative stress values and reported with standard error. The maximum strain for each sample was then defined as the strain at the maximum tested stress of 250 kPa.

### Suture Retention Testing

The suture retention test has been previously described (Freytes D O, et al. Effect of storage upon material properties of lyophilized porcine extracellular matrix derived from the urinary bladder. Journal of Biomedical Materials Research Part B-Applied Biomaterials. 2006; 78B(2): 327-33). The suture retention strength was performed according to ANSI/AAMI VP20-1994 Guidelines for Cardiovascular Implants-Vascular Prostheses. The suture retention strength was defined as the force required to pull a suture through the full thickness of the material. A 2-0 Prolene suture with a SH taper needle was passed through the test article with a 2-mm bite depth using a simple suture technique. The specimen was clamped at one end while the suture was attached to the uniaxial mechanical testing machine (Instron Model 3345 single column materials testing system) and pulled at a constant rate of 10 cm/min according to the aforementioned standard. Two tests were performed 1.5 cm apart on the same edge of the test article and the maximum load was recorded for each test.

Rheologic Testing of coECM Hydrogels

The rheological characteristics of coECM hydrogels at 4 and 8 mg/mL were determined with a rheometer (AR2000, TA instruments, New Castle, Del.) operating with a 40 mm parallel plate geometry. The temperature was controlled within 0.1° C. using a Peltier plate. Pre-gels were pH neutralized on ice and were immediately loaded onto the rheometer plate pre-cooled to 10° C. Mineral oil was spread along the edge (i.e. the free surface of the hydrogel) to minimize evaporation. After loading, the steady shear viscosity was measured by applying a stress of 1 Pa at a frequency of 0.159 Hz. The temperature was then increased to 37° C. to induce gelation and a small amplitude oscillatory strain of 0.5% was imposed to track the gelation kinetics. After complete gelation, a creep test (1 Pa for 20 s) was performed to verify that there was no slip between the ECM hydrogels and rheometer plates.

### Turbidometric Gelation Kinetics

The gelation kinetics of coECM hydrogels was evaluated turbidometrically. Briefly, neutralized pre-gel solutions of coECM at 4 and 8 mg/mL concentrations were prepared on ice. For each ECM concentration, 100 μL/well was added to a 96-well plate and placed into a plate reader (Spectramax M2, Molecular Devices, Sunnydale, Calif.) pre-warmed to 37° C. Absorbance at 405 nm was read every 2 min for 60 min and the readings were scaled from 0 (initial absorbance) to 100% (maximum absorbance). The time to half gelation (t_{1/2}) was defined as the time at 50% absorbance. Gelation rate was defined as the slope of the linear region of the gelation curve. The lag time (t_{lag}) was defined as the intercept of the linear region of the gelation curve with 0% absorbance.

### In Vitro Cytocompatibility

In vitro cytocompatibility was determined using a LIVE/DEAD Viability/Cytotoxicity Kit

(Invitrogen) following the manufacturer's directions. Briefly, 1 multilaminates of colonic ECM (coECM), cross linked colonic ECM (XL-coECM), or scraped native colon were sterilized with ethylene oxide. 8 mg/ml coECM hydrogels were prepared as previously described. Intestinal epithelial cells (IEC6; ATCC) were cultured and maintained in complete growth media consisting of 5% fetal bovine serum (FBS) (Gibco), 0.1 U/ml bovine insulin, 100 ug/ml penicillin, 100 U/ml streptomycin. IECs were seeded at 1×10⁶ cells/scaffold for 48 h. Cell viability was compared to growth on tissue culture plastic (TCP). Cells were stained with 4 mM green fluorescent calcein-AM (cAM) and 2 mM red fluorescent ethidium homodimer-1 (EthD-1) to detect viable and dead cells, respectively. Images were taken with a Zeiss Axiovert microscope capturing 3 random fields across the scaffold. Quantification of percentage of live and dead cells was completed using a custom CellProfiler pipeline. Cell-seeded scaffolds were then fixed in 2% paraformaldehyde and formalin, embedded in paraffin, and sectioned for hematoxylin and eosin (H&E) staining.

### In Vitro Macrophage Response

Primary murine bone marrow derived macrophages were isolated as described previously (Sicari B M, et al. The promotion of a constructive macrophage phenotype by solubilized extracellular matrix. Biomaterials. 2014: 35(30): 8605-12). Briefly, bone marrow was isolated from the femur and tibia of C57bl/6 mice and cultured for 7 days in 100 ng/ml MCSF to derive naïve (MΦ) macrophages. Macrophages were then activated with 20 ng/ml IFNγ and 100 ng/ml lipopolysaccharide (LPS) to derive M1 macrophages, 20 ng/ml IL-4 to derive M2 macrophages, or 200 ug/ml of solubilized colonic ECM for 18 hours. Macrophages were then fixed with 2% paraformaldehyde for immunolabeling or lysed for western blot analysis. Cells were incubated in blocking buffer consisting of 0.1% Triton-X 100, 0.1% Tween-20, 2% bovine serum albumin (BSA), and 4% goat serum for 1 hour at room temperature. Following blocking, cells were incubated in the following primary antibodies diluted in blocking buffer for 16 h at 4° C.: (1) anti-F4/80 (abeam) at 1:200, (2) anti-iNOS (abeam) at 1:100, or anti-RELMα (Fizz1, Peprotech) at 1:200. Cells were washed with phosphate buffered saline (PBS) and incubated in secondary antibodies diluted in blocking solution for 1 hour at room temperature: (1) Alexa Fluor 488 goat anti-rat at 1:200, (2) AlexaFluor 488 donkey anti-rabbit at 1:200. Cells were then washed with PBS and counterstained with DAPI nuclear stain. The assay was completed on four separate days (n=4) and cells were imaged using a Zeiss Axiovert microscope with exposure times standardized using classically polarized (IFNy/LPS or IL-4) internal controls. Percentage of F4/80, iNOS, and Fizz1 positive cells were quantified using CellProfiler.

Western blotting was performed to analyze an additional marker of M2 macrophages. Cells were lysed and lysates were boiled at 95° C. for 5 minutes and loaded at 100 ug/well in a 4-20% gradient polyacrylamide SDS page gel. Separated proteins were transferred to PVDF membranes using a wet-transfer set up and incubated for 16 hours in 3% milk, TBS-T to prevent non-specific antibody binding. Membranes were incubated in the following primary antibodies for 18 h in 3% milk at 4° C.: (1) polyclonal anti-rabbit mannose receptor (Abeam, Cambridge, Mass.) at 1:714 dilution for an M2 marker or (2) monoclonal anti-mouse β-actin (Santa Cruz, Dallas, Tex.) at a dilution of 1:1000 as a loading control. Three blots completed on separate days (n=3) and were visualized using a LICOR Odyssey fluorescent imaging scanner. Densitometry of protein expression was standardized to the loading control.

### In Vivo Cytocompatibility

### Abdominal Wall Defect Model

The partial thickness abdominal wall defect model for evaluation of the host response to biomaterials is well established. Surgical plane of anesthesia was achieved via inhalation of 2% isoflurane in oxygen. The surgical site was prepared by shaving the lateral abdominal region on both sides of each animal, scrubbing, and draping. Animals were placed in a lateral decubitus position and incisions were made along the midaxillary line. The skin and subcutaneous tissues medial to the incision were separated from the underlying muscle tissues. A 1.5 cm by 1.5 cm section of the external and internal oblique layers of the ventral lateral abdominal wall were excised while the underlying transversalis fascia and peritoneum were left intact. The muscle defect was subsequently repaired with a size-matched piece of the chosen test article or a hydrogel. The test articles were secured in place with 4-0 Prolene at each of the four corners securing the device to the surrounding and underlying musculature allowing for mechanical loading of the test article during the normal abdominal wall activity of daily living, and facilitating identification at the time of explantation. Incisions were closed with 4-0 Vicryl sutures. Animals were recovered from anesthesia, returned to the housing unit, and received 0.02 rug Buprenex (buprenorphine hydrochloride) by subcutaneous injection the day of surgery and for two additional days twice daily. Baytril (20 mg) was administered orally the day of surgery and for two additional days. The dietary habits, general health status, and the surgical site were monitored daily and recorded. The implant site containing test articles and surrounding adjacent tissue were isolated and placed in 10% neutral buffered formalin (NBF). Samples were then embedded in paraffin and cut into 6 μm sections for histologic studies.

### Histomorphologic Scoring

Tissue sections were stained with hematoxylin and eosin (H&E) for qualitative and semiquantitative histomorphologic analysis of remodeling outcomes. Two blinded investigators scored sections according to an established semi quantitative scoring method as shown in Table 1. Scoring criteria were used to group devices according to the following categories: chronic inflammation and foreign body reaction (quantitative score<5), early inflammatory cell infiltration with decreased cellularity and little evidence of constructive downstream remodeling (5≤quantitative score<10), and early infiltration by inflammatory cells and signs of constructive remodeling at later time points (quantitative score>10).

### Host Response: Macrophage Immunolabeling

To characterize macrophage phenotype following ECM implantation, paraffin-embedded tissue sections were deparaffinized. Heat-mediated antigen retrieval was performed in heated citrate buffer for 20 minutes (10 mM citrate, pH 6.0 at 95-100° C.). Tissue sections were allowed to cool and were incubated in blocking solution consisting of 2% goat serum, 1% bovine serum albumin (BSA, Sigma), 0.1% Triton X-100 (Sigma), and 0.1% Tureen-20 (Sigma) in PBS to prevent non-specific antibody binding. After blocking, tissue sections were incubated with primary antibodies diluted 1:150 in blocking solution overnight at 4° C. CD68 (mouse anti-rat CD68 clone ED1, AbD Serotec) was used as a pan-macrophage marker, CD86 (rabbit anti-human CD86, clone EP 1158Y, abeam) was used as an M1 macrophage marker, and CD206 (goat anti-human CD206 polyclonal, Santa Cruz) was used as an M2 marker. Following primary incubation, sections were washed in PBS and incubated in the following fluorescently conjugated secondary antibodies for 1 hour at room temperature diluted in blocking solution: AlexaFluor donkey anti-mouse 594 at 1:200 (Invitrogen), PerCP-Cy5.5 donkey anti-rabbit at 1:300 (Santa Cruz), and AlexaFluor donkey anti-goat 488 at 1:200 (Invitrogen). Tissue sections were washed, counterstained with 4′,6-diamindino-2-phenylindole (DAPI), and coverslipped. Multispectral images were acquired with appropriate filter sets using a Nuance microscope and spectrally unmixed to remove tissue auto-fluorescence. Four images were taken along the defect and underlying transversalis interface at 200× magnification. Total cells expressing CD68 and either CD86 or CD206 were quantified using CellProfiler image analysis software. Macrophages were defined as CD68 positive colocalized with nuclei. M1 and M2 cells were defined as macrophages (CD68+) coexpressing CD86 or CD206 respectively. Cells expressing both CD86 and CD206 were subtracted from the M1 and M2 totals, and an M2:M1 ratio was calculated for each image.

### Statistical Analysis

A two-tailed equal variance student's t-test was used to determine whether the DNA, phospholipid, GAGs, HA, collagen, growth factor, and mechanics of the coECM were different than that of native colon (p<0.05). A t-test was also used to determine differences in turbidometric and rheologic properties of 4 mg/mL vs. 8 mg/mL coECM hydrogels. A one-way analysis of variance (ANOVA) with post-hoc Tukey test was used to determine differences in the percentage of viable cells in culture, percentage of cells expressing macrophage phenotype markers, in-vivo histologic scores, and in-vivo macrophage phenotype ratio. All data are reported as mean±standard error.

### Results

### Decellularization Efficacy

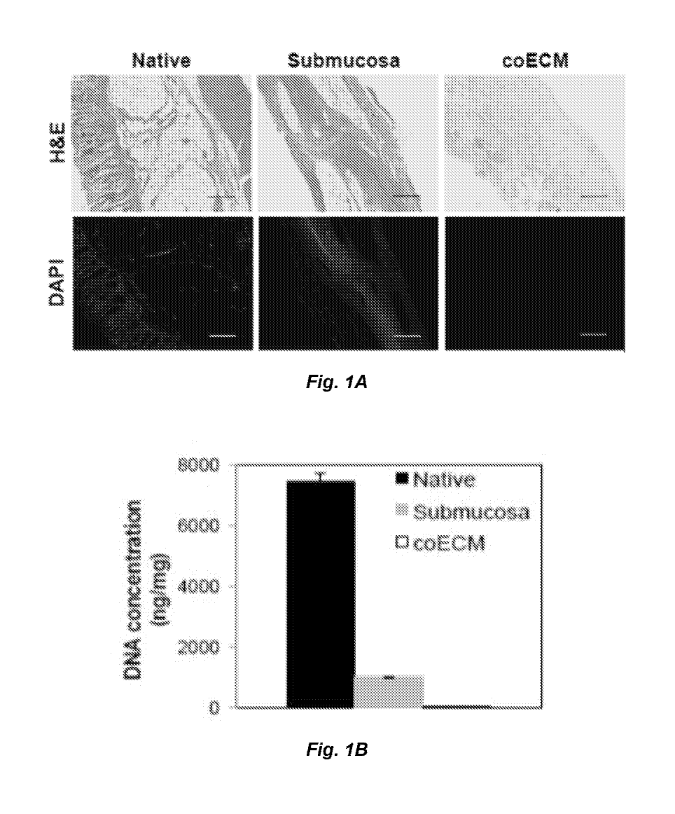

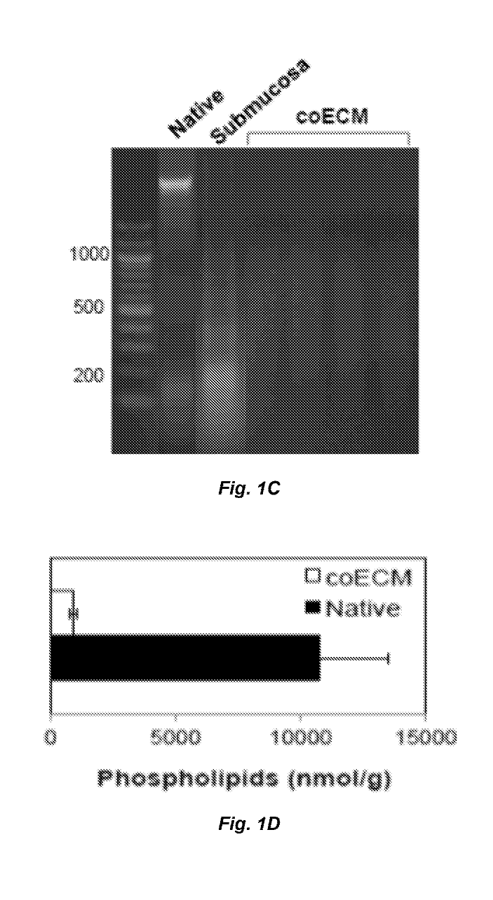

A protocol for effective decellularization of colonic submucosa was identified with the use of enzyme and detergent washes. Although previously described decellularization protocols for gastrointestinal tissue (e.g., esophageal and small intestine) do not require delipidization, the porcine colonic submucosa had high lipid content (FIG. 1A) and thereby required delipidization for effective decellularization. The degree of decellularization following the described method was assessed using previously established guidelines for decellularization (Freytes D O, et al. Journal of Biomedical Materials Research Part B-Applied Biomaterials. 2006; 78B(2): 327-33). No intact nuclei were visible by H&E or DAPI staining following decellularization (FIG. 1A). The concentration of remnant DNA in coECM (43±5.3 ng/mg) was markedly less (p<0.001) than that in native colonic tissue (7435±420 ng/mg) and native submucosa (998±31 ng/mg) (FIG. 1B). Residual DNA was present in fragments less than 200 bp in length (FIG. 1C). In addition to DNA content, phospholipid concentration in the coECM was used as an indicator of decellularization efficacy. The concentration of phospholipids, fundamental components of cell membranes, in the coECM was 876±105 nmol/g and was much lower (p<0.001) than the native colon (FIG. 1D).

Biochemical and Structural Properties of coECM

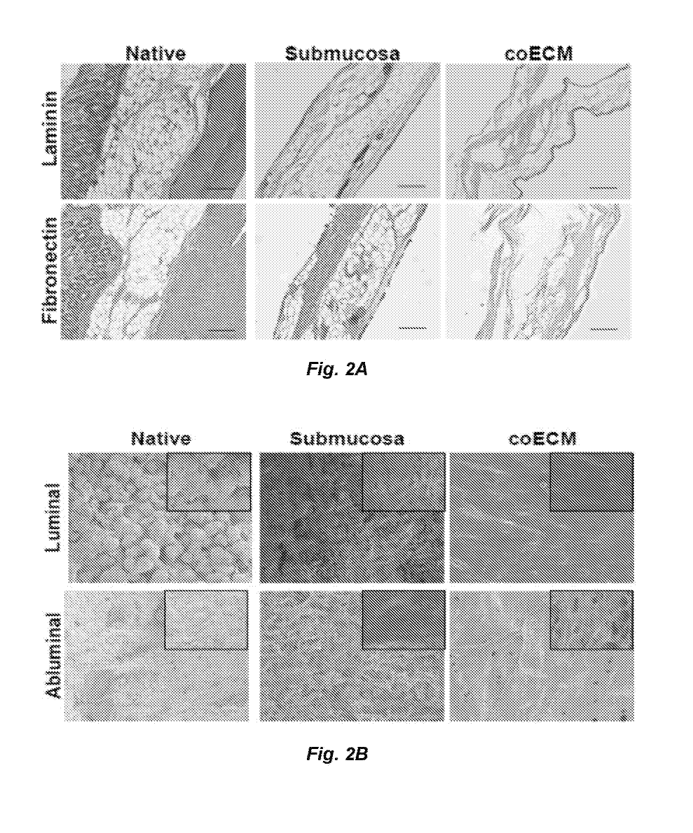

The preservation and spatial distribution of ECM proteins, including basement membrane-associated laminin, and a non-basement membrane protein, fibronectin, were examined. Immunolabeling expectedly showed that laminin was present along the basement membrane of the native colon but this layer was mechanically removed during decellularization and thus laminin was largely absent in the coECM. Similarly, positive staining for fibronectin was present and distributed throughout the native colonic tissue but only diffuse staining for fibronectin was observed in the coECM (FIG. 2A). The surface ultrastructure of the coECM scaffold was observed with SEM. SEM images of the luminal and abluminal surface of coECM showed a smooth surface on the luminal surface of the coECM. The abluminal surface, however, had a more textured and fibrous appearance (FIG. 2B). Thus, the micro- and ultrastructure of the scaffold is unique and a “sidedness” exists such that the luminal surface is smoother than the abluminal surface.

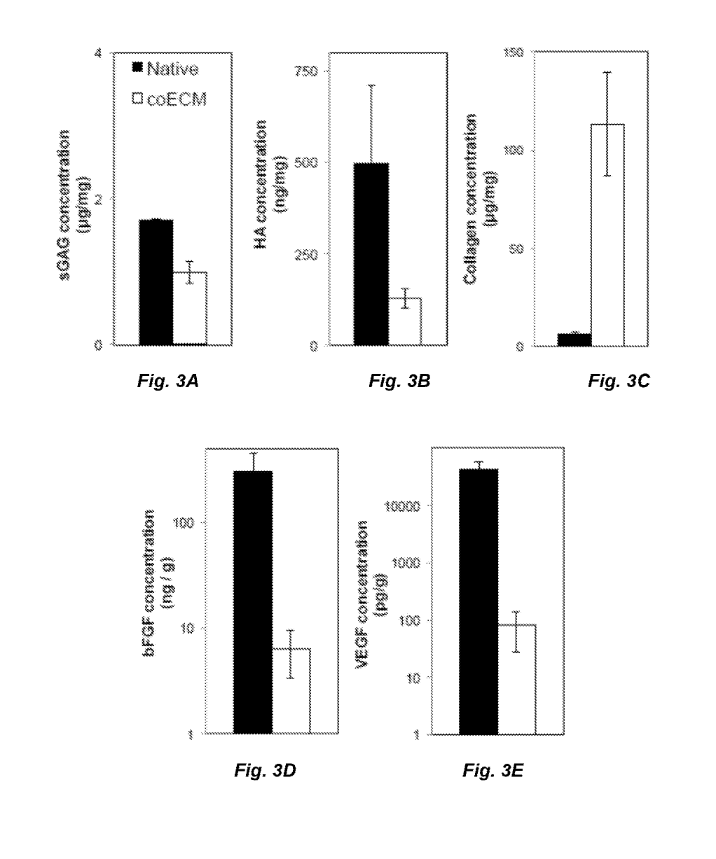

Biochemical characterization of coECM showed that important ECM constituents are present in the decellularized colonic mucosa. GAGs, both sulfated (sGAGs) and non-sulfated, were retained in coECM. A large percentage of sGAGs were preserved in the coECM although the concentration was less than (p=0.012) the native tissue (FIG. 3A). Hyaluronic acid, a non-sulfated GAG, was present in the coECM while the concentration was also lower (p=0.001) than native tissue (FIG. 3B). The amount of fibrilliar collagen in the coECM was expectedly greater (p=0.039) than native tissue as collagen represents a large proportion of the ECM (FIG. 3C). Lastly, although present in reduced levels compared to native tissue, both bFGF (FIG. 3D, p<0.001) and VEGF (FIG. 3E, p<0.001) were retained in the coECM. The biochemical characteristics of the coECM is unique. Both sulfated and non-sulfated glycosaminoglycans remain following decellularization. Growth factors also are retained.

Mechanical Properties of coECM Scaffold

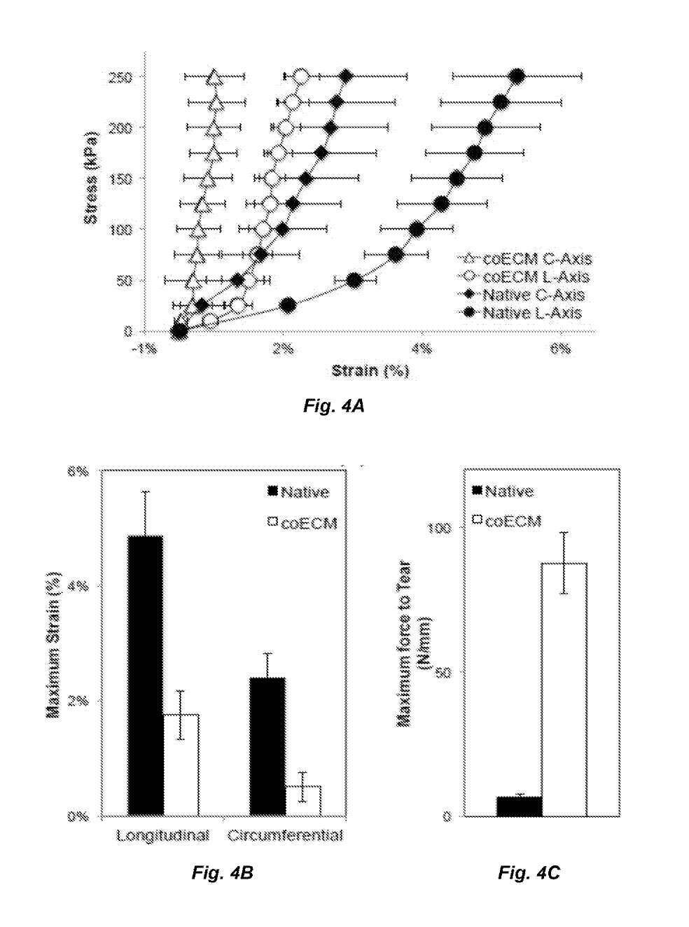

The equibiaxial stress response of the native colon showed anisotropic behavior with a maximum strain of 4.9% and 2.4% in the longitudinal and circumferential direction, respectively (FIG. 4A and 4B). The coECM showed similar anisotropy, but had a lower compliance along both the longitudinal (1.8%, p=0.042) and circumferential (0.5%, p=0.023) axes (FIGS. 4A and 4B). The multilaminate coECM scaffold, however, had marked increase (p<0.001) in suture retention strength compared to the native colon (FIG. 4C). Mechanical properties of the scaffold show anisotropy with increased compliance in the longitudinal direction.

Rheologic and Turbidometric Properties of coECM Hydrogel

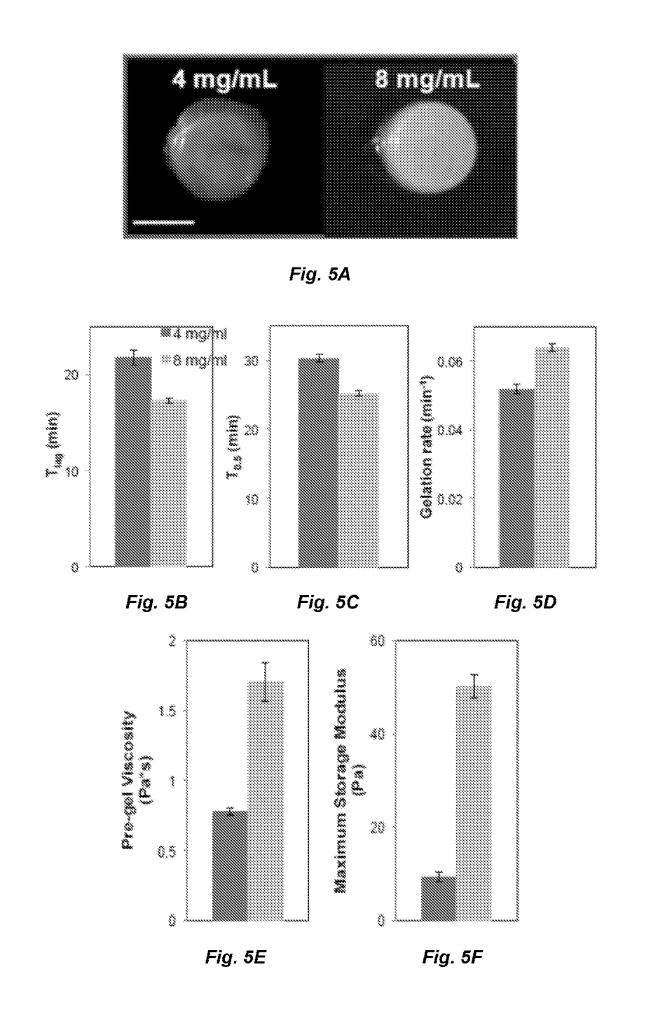

The turbidometric and rheological properties of the coECM hydrogel were concentration dependent. Macroscopically, the higher ECM concentration 8 mg/mL hydrogels had a rigid structure with defined edges, and could be handled and manipulated with forceps while the 4 mg/mL hydrogels were softer with rounded edges and not easily handled (FIG. 5A). Compared to the 4 mg/mL hydrogel, the more concentrated 8 mg/mL hydrogel had a shorter lag time (17±0.3 vs. 22+0.8 min; p<0.001) prior to gelation (FIG. 5B), and gelled more rapidly (FIG. 5C and 5D). Results of rheological testing showed that the 8 mg/mL pre-gel was more (p=0.027) viscous (FIG. 5E) and the hydrogel that formed was much stiffer (p=0.001) than the 4 mg/mL hydrogel (FIG. 5F). Rheologic properties of the ECM are unique, and are concentration-dependent.

In Vitro Cell Response to coECM

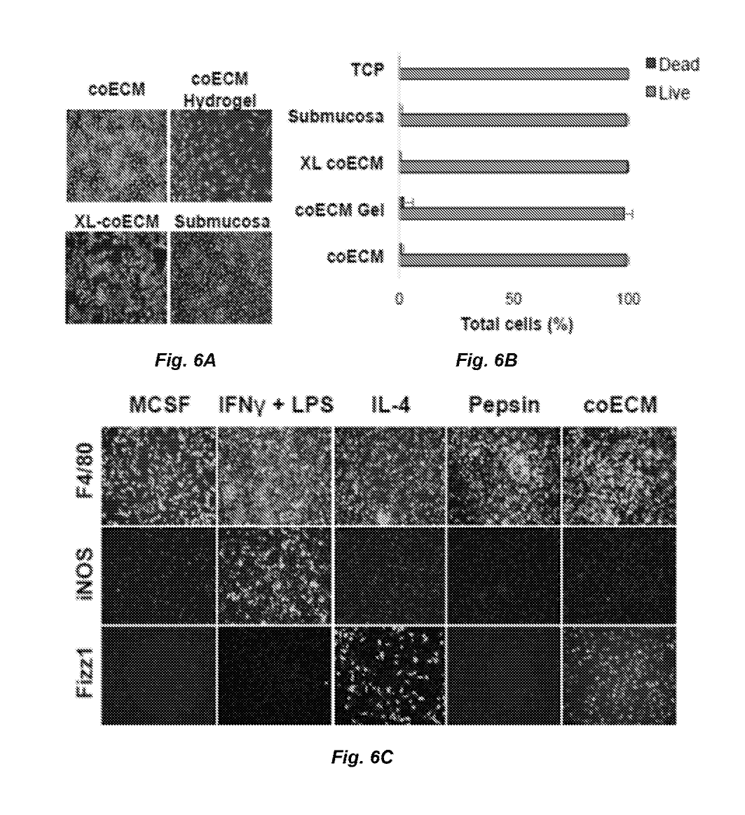

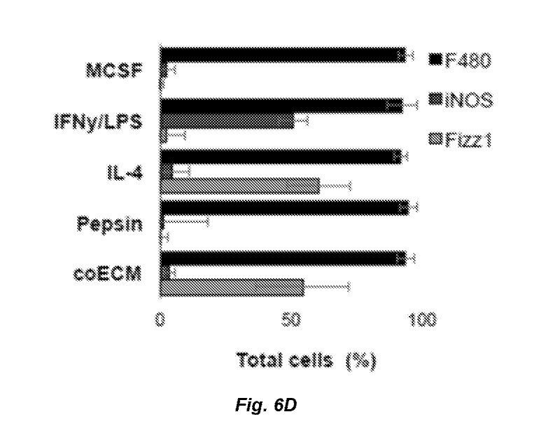

Intestinal epithelial cells retained nearly 100% viability when seeded on coECM, XL-coECM, coECM gel, and submucosa (FIG. 6A). There was no difference between these treatments and when compared to tissue culture plastic after 24 hours in culture (FIG. 6B).

Primary murine bone marrow derived macrophages were activated to the M1 (IFNγ/LPS) and M2 (IL-4) phenotypes for 18 hours or treated with 200 μg/ml of solubilized coECM. All experimental groups showed uniform F4/80 staining with 93.4±0.5% of cells expressing the pan-macrophage marker. The controls showed an expected increase (p<0.001) in iNOS when macrophages were treated with IFNγ/LPS and an increase (p<0.001) in Fizz1 when treated with IL-4. The coECM treatment was found to promote M2-like macrophage activation, similar to IL-4 treated macrophages as shown by Fizz1 expression accompanied by little iNOS expression (FIG. 6C). Results quantified using CellProfiler show a large Fizz1+ cell population and small iNOS+cell population when treated with coECM, suggesting that coECM directly promotes a constructive, M2-like macrophage phenotype (FIG. 6D). The presence of CD206, a cell surface receptor that is indicative of M2 macrophage phenotype, was assessed using western blot analysis and normalized to a β-actin loading control. Similar to above with Fizz1 expression, the ratio of CD206:β-actin was on average greatest in macrophages following IL-4 (1.8±0.4) and coECM treatment (1.2±0.2) compared to IFNy/LPS treatment (0.6±0.2) or the pepsin control, although not significant. Thus, coECM scaffold and coECM hydrogel support growth of intestinal epithelial cells and promote an anti-inflammatory macrophage phenotype in vitro.

### In Vivo Host Response

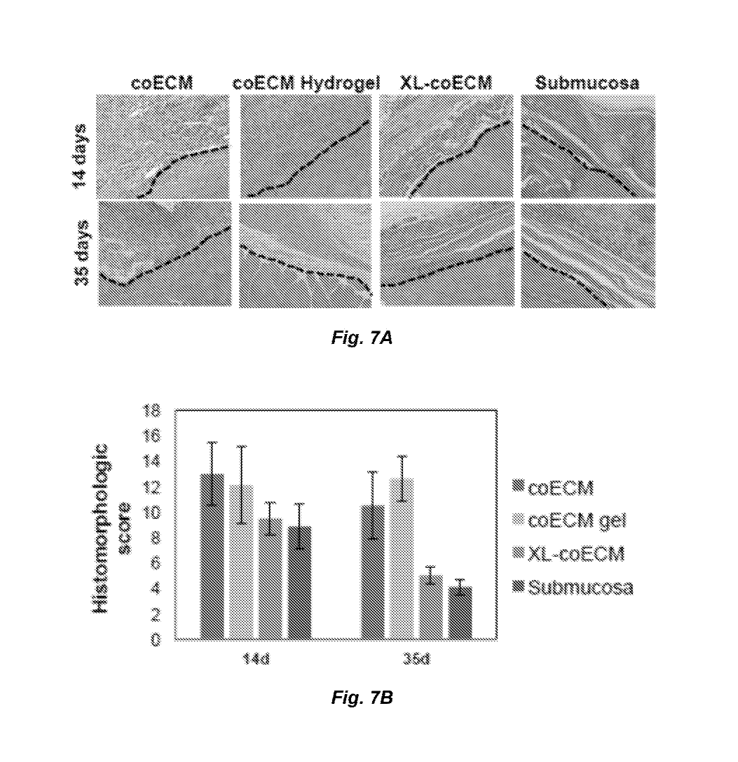

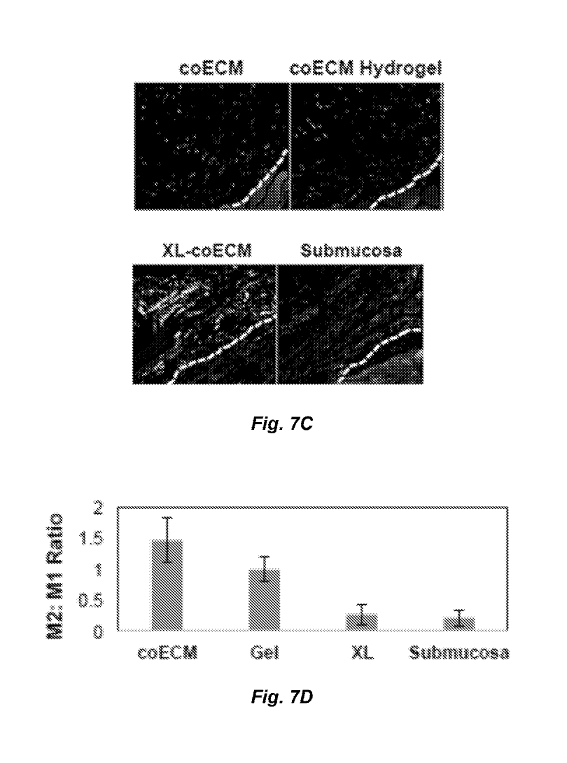

The in vivo host response to coECM was examined in a rat abdominal wall defect model at both 14 and 35 days following implantation. In addition to coECM and coECM hydrogel, two additional groups were used as negative controls. Native colonic submucosa was used as a control to validate the necessity for effective decellularization. Cross-linked coECM was used to validate the need for scaffold degradation. By 14 days, coECM sheet and gel implants showed histologic evidence of a robust cell infiltrate and partial scaffold degradation shown by hematoxylin and eosin staining (FIG. 7A) with an average histologic score of 12.9 and 12.1, respectively (FIG. 7B). In contrast, cross-linked coECM and native colonic submucosa implants were characterized by very little cellular infiltration and vessel formation and minimal scaffold degradation (FIG. 7A). Disorganized connective tissue was present along the interface of XL-coECM and submucosa with the native underlying muscle. The average histologic scores for XL-coECM and native submucosa was 9.4 and 8.8, respectively (FIG. 7B), which were both significantly less (p<0.05) than the coECM and coECM hydrogel. By 35 days, coECM sheets and gels were completely degraded while XL-coECM and native colonic submucosa remained almost completely intact (FIG. 7A). The histologic scores for both coECM and coECM hydrogel were greater (p<0.05) than both XL-coECM and native submucosa (FIG. 7B).

Macrophage immunolabeling at 7 days post-surgery (FIG. 7C) showed a predominant CD68+CD206+ M2 macrophage population in coECM sheet and gel treated groups when compared to a predominant CD68+CD86+ proinflammatory M1 macrophage phenotype following XL-coECM or colonic submucosa implantation as shown in FIG. 7C. The ratio of the M2:M1 macrophages in the coECM scaffold was 1.46±0.3 which was greater (p<0.01) than the XL-coECM and native submucosa (FIG. 7D). The M2:M1 ratio in the coECM gel was on average also greater than the XL-coECM and native submucosa, although not significant (p=0.055). Thus, coECM scaffold and coECM hydrogel are biocompatible and support an anti-inflammatory immune cell population in vivo.

### Discussion

Functional replacement of injured or missing GI tissue requires a diverse tool set to promote the growth and differentiation of specialized cell types and tissue layers that vary from esophagus to small intestine to colon. The present study represents a thorough characterization of an ECM bioscaffold derived from porcine colon (coECM). The coECM scaffold was shown to be decellularized—meeting previously established stringent criteria (Crapo P M, et al. An overview of tissue and whole organ decellularization processes. Biomaterials. 2011; 32(12): 3233-43). The decellularization protocol effectively removed native DNA while preserving essential structural and biochemical ECM components including sGAGs, hyaluronic acid, collagen, bFGF, and VEGF. The coECM scaffold was shown to retain similar mechanical properties and anisotropy as native colon. In vitro and in vivo coECM is cytocompatible and promotes a constructive, M2-like macrophage phenotype when compared to its ineffectively decellularized or cross-linked counterparts. Such properties make coECM promising for use as an “off-the-shelf” gastrointestinal repair biomaterial.

Regions of the GI tract, specifically the small intestinal submucosa (SIS) and esophageal mucosa, have been successfully decellularized previously. Just as the native GI tract is a highly complex and variable organ, the composition and properties of each of these bioscaffolds are also variable. Such properties are largely dependent on the method of decellularization utilized and the source tissue from which they are derived. SIS-ECM is prepared primarily by mechanical delamination and exposure to peracetic acid. Esophageal ECM (eECM), on the other hand, is exposed to a series of enzymatic and chemical detergent treatments after mechanical delamination methods, similar to coECM preparation though an additional delipidization step is necessary for coECM decellularization. Each of these protocols results in scaffolds with unique properties and compositions. For example, when compared to eECM, coECM has a lower maximum strain along the longitudinal axis (Keane T J, et al. Preparation and characterization of a biologic scaffold from esophageal mucosa. Biomaterials. 2013; 34(28): 6729-37). Differences in these values could be important determinants of in vivo remodeling outcomes. Previous studies have shown that tensile strength increases from proximal to distal intestine (Watters D A, et al. Mechanical properties of the rat colon: the effect of age, sex and different conditions of storage. Q J Exp Physiol. 1985; 70(1): 151-62), likely stemming from the need of distal colon to accommodate changes in higher stress as fecal pellets become more solid. Distinguishing the mechanics of different regions of the gut and a thorough comparison and understanding of the similarities and differences between ECMs derived from different source tissues could have important implications for application-specific selection of bioscaffolds, particularly in gastrointestinal repair applications which are also inherently diverse and complex and require individualized mechanics for peristalsis, digestion, absorption, and gastric motility.

While heterologous ECM bioscaffolds have been used with success in multiple anatomic locations for constructive tissue remodeling, a subset of studies have indicated that it may be advantageous to utilize site-specific ECM (Sellaro T L, et al. Maintenance of human hepatocyte function in vitro by liver-derived extracellular matrix gels. Tissue Eng Part A. 2010; 16(3): 1075-82; Sellaro T L, et al. Maintenance of hepatic sinusoidal endothelial cell phenotype in vitro using organ-specific extracellular matrix scaffolds. Tissue Eng. 2007; 13(9): 2301-10; Cheng NC, et al. Chondrogenic differentiation of adipose-derived adult stem cells by a porous scaffold derived from native articular cartilage extracellular matrix. Tissue Eng Part A. 2009; 15(2): 231-41; Cortiella J, et al. Influence of acellular natural lung matrix on murine embryonic stem cell differentiation and tissue formation. Tissue Eng Part A. 2010; 16(8): 2565-80; and Zhang Y, et al. Tissue-specific extracellular matrix coatings for the promotion of cell proliferation and maintenance of cell phenotype. Biomaterials. 2009; 30(23-24): 4021-8). Each tissue has a distinct composition of ECM in which the appropriate signaling molecules and structural components are present to allow for cell growth and differentiation and synergized tissue function. It is reasonable to assume, therefore, that decellularization of site-specific tissue would provide the optimal inductive template for tissue engineering in its respective anatomic location. Whether or not site-specific ECM use is relevant in all therapeutic applications, however, is not fully understood. Results of the present study show coECM facilitated constructive tissue remodeling in a heterologous location. Design of a tissue-engineered intestine should take into consideration gut function such as contractility. Biomaterials for gut replacement should allow for regeneration of functional muscle and directional self-organization of functionally distinct layers that perform a variety of functions including nutrient absorption, mucus secretion, and motility. Future studies are warranted to determine the efficacy of coECM as a bioscaffold in gastrointestinal, or specifically colon, disease/injury models.

Although the mechanism(s) of action of ECM-mediated tissue remodeling are only partially understood, the activation/polarization of infiltrating macrophages at the remodeling site from a pro-inflammatory, cytotoxic M1 phenotype to an immunoregulatory, constructive M2 macrophage phenotype has been shown to be a predictor of favorable downstream remodeling outcomes. The present study shows that coECM promotes a predominant M2 (CD68+CD206+) macrophage phenotype when compared to native colonic submucosa and XL-coECM following implantation. The immunomodulatory properties of coECM may prove beneficial in cases of inflammatory bowel disease treatment in which it is postulated that the host lamina propria macrophages fail to polarize toward a more tolerant M2-like phenotype. The present study shows that coECM can also be prepared in a hydrogel form with unique and concentration-dependent viscoelastic properties, providing flexibility for in vivo applications such as injectable or enema administration.

Biologic scaffolds are selectively preferred over synthetic scaffolds for many tissue repair applications because of their degradability in vivo. It is now well accepted that cross-linking biologic scaffolds results in slower degradation and often encapsulation and fibrosis. Such inhibition of scaffold degradation prevents the release or exposure of matricryptic peptides and is consistently associated with less than desirable outcomes (Valentin J E, et al. Extracellular matrix bioscaffolds for orthopaedic applications. A comparative histologic study. J Bone Joint Surg Am. 2006; 88(12): 2673-86 and Valentin J E, et al. Functional skeletal muscle formation with a biologic scaffold. Biomaterials. 2010; 31(29): 7475-84). H&E staining of abdominal wall explants shows that coECM sheet and hydrogel formulations are characterized by a robust cellular infiltrate at 14 days and are largely degraded by 35 days, unlike the native (non-decellularized) submucosa and XL-coECM which were characterized by mostly disorganized connective tissue and some encapsulation as reflected by a lower histomorphologic score. This score differential is likely due to the incomplete decellularization of the submucosa graft and the inability of XL-coECM to degrade. Ineffective decellularization has been shown to be a crucial factor in provoking a foreign body reaction from the host following bioscaffold implantation (Keane T J, et al. Consequences of ineffective decellularization of biologic scaffolds on the host response. Biomaterials. 2012; 33(6): 1771-81 and Keane T J, et al. Methods of Tissue Decellularization Used for Preparation of Biologic Scaffolds and In-vivo Relevance. Methods. 2015). Degradation products of coECM have been shown to not only promote a predominant F480±/Fizz1+ macrophage population in vitro, but previous work has also shown that degradation products of ECM are chemotactic and mitogenic for progenitor cells both in vitro and in vivo (Reing J E, et al. Degradation products of extracellular matrix affect cell migration and proliferation. Tissue Eng Part A. 2009; 15(3): 605-14; Sicari B M, et al. The promotion of a constructive macrophage phenotype by solubilized extracellular matrix. Biomaterials. 2014; 35(30): 8605-12; and Vorotnikova E, et al. Extracellular matrix-derived products modulate endothelial and progenitor cell migration and proliferation in vitro and stimulate regenerative healing in vivo. Matrix Biol. 2010; 29(8): 690-700). These results emphasize the importance for effective decellularization and scaffold degradation.

### Conclusion

A biologic scaffold was successfully prepared from porcine colon. The coECM scaffold was effectively decellularized and retained important ECM constituents. The decellularized tissue was prepared in hydrogel or lyophilized sheet forms to address diverse gastrointestinal repair applications. Both forms of ECM were conducive to intestinal epithelial cell growth and were shown to promote a constructive macrophage phenotype in-vitro. Surgically implanted coECM scaffold and hydrogel also promote an immunomodulatory host response and site appropriate tissue deposition.

### EXAMPLE 2

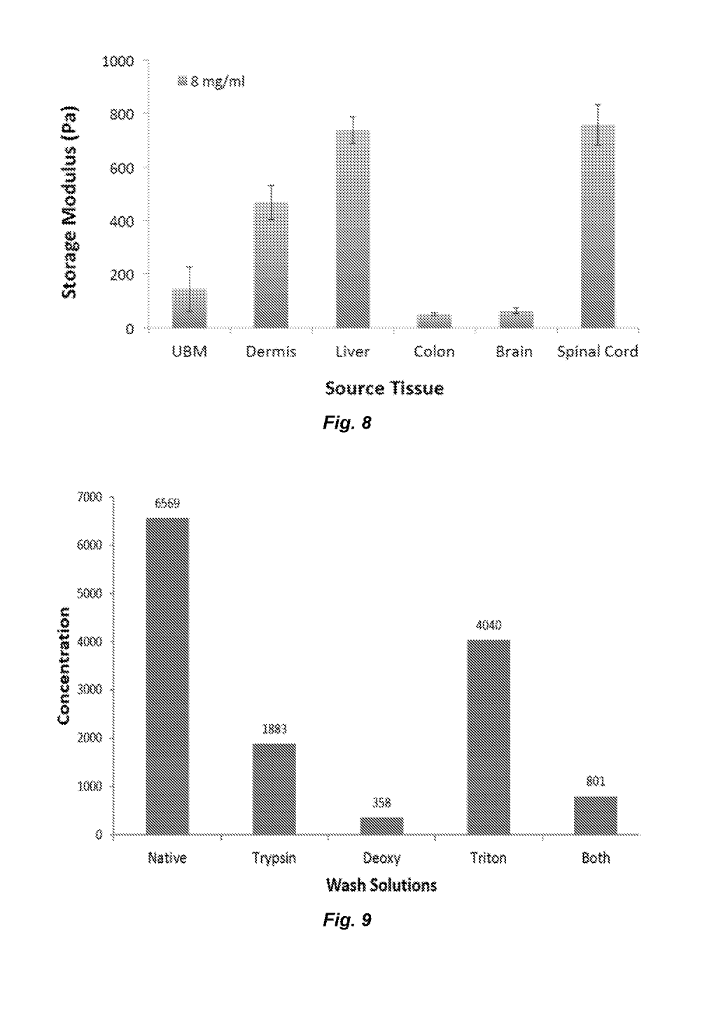

FIG. 8 is a graph showing storage modulus of various hydrogels from varying source tissues, comparing rigidity of 8 mg/ml hydrogels from varying source tissues.

### EXAMPLE 3

Lyophilized colon were placed in a 2 L flask with a 2:1 (v/v) solution of chloroform methanol and placed in the fume hood with a stir bar for 30 minutes. Following chloroform step, solution was drained and 100% ethanol was added, placed in a shaker at 300 RPM for 5 minutes, this was repeated 3 times. Subsequently, followed by a series of graded ethanol washes: 3 washes of 90% ethanol, 3 washes of 70% ethanol at 300 RPM. Ethanol was drained and three water washes at 300 RPM were performed. Decellularization of colon proceeded by rehydration of the colon with 0.02% Trypsin/EDTA, incubated in a water bath for 1 hour at 37° C. Colon was then split into four groups: Trypsin/EDTA only, Trypsin/EDTA followed by 3% Triton X-100 for 1 hour on shaker at 300 RPM, Trypsin/EDTA followed by 4% Deoxycholic acid for 1 hour on shaker at 300 RPM, and Trypsin/EDTA followed by 3% Triton X-100 for 1 hour followed by 4% Deoxycholic acid for 1 hour. Colon were stored at 4° C. until disinfection. Disinfection was done with PAA (peracetic acid)

### EXAMPLE 4

The amount of remaining nucleic acid was measured to assess the best decellularization protocol. DNA was extracted from colon using a proteinase K, phenol/chloroform extraction method. Colon that were either: 1) not decellularized, that is native colon, 2) decellularized with Trypsin/EDTA, 3) deoxycholic acid, 4) Triton X-100, or 5) with both deoxycholic acid and Triton X-100 were processed and assessed for nucleic acid content.

As seen in FIG. 9, the deoxycholate decellularization protocol worked the best. From the data, the Deoxycholate removed cells better than the decellularization protocol with both the deoxycholate and triton detergents. Logically, one would assume that the decellularization method with two detergents would have fewer cells than either of the detergents alone. However, results here prove otherwise. This suggests that the addition of Triton with deoxycholic acid does not substantially reduce the amount of remnant DNA.

### EXAMPLE 5

The amount of remaining nucleic acid was measured to assess the best decellularization protocol. DNA was extracted from colon using a proteinase K, phenol/chloroform extraction method.

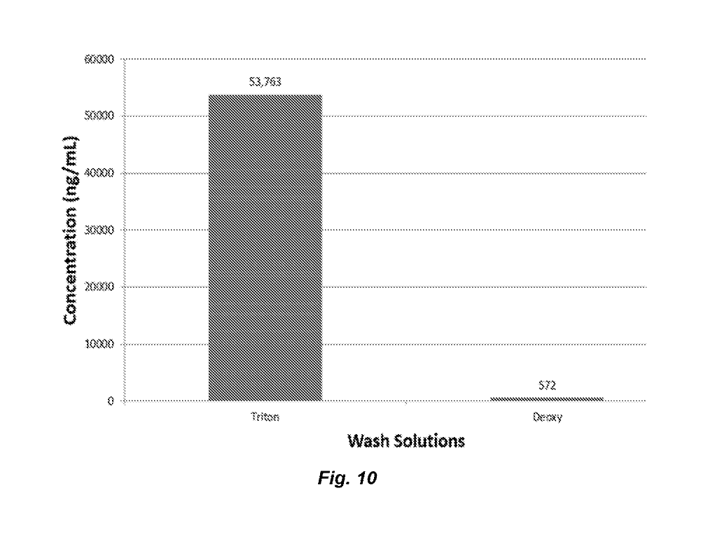

Colon were either decellularized using 4% Deoxycholate or 3% Triton. As shown in FIG. 10, the tissue decellularized by 4% Deoxycholate removed more DNA than the 3% Triton decellularization process.

### EXAMPLE 6

Colon were decellularized by the following two methods: 1) Colons were washed with 4% deoxycholate for 30 minutes at 300 RPM, followed by two washes with water for 5 minutes each at 300 RPM, and then another wash with 4% deoxycholate for 30 minutes at 300 RPM, or 2) colons were washed with 3% Triton at 300 RPM for 30 minutes, followed by two washes with water for 5 minutes, then washed with 4% deoxycholate for 30 minutes, followed by two washes with water for 5 minutes, then washed with 3% triton for 30 minutes, followed by two washes with water for 5 minutes, followed by a wash with 4% deoxycholate for 30 minutes, and then finally followed by two washes with water for 5 minutes.

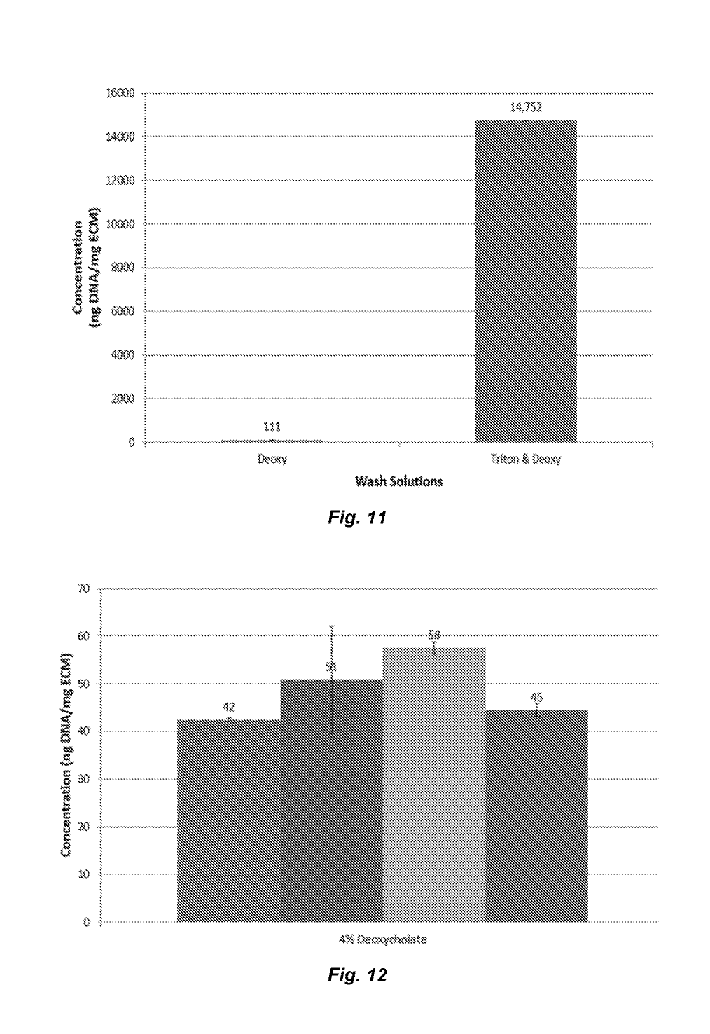

DNA was extracted and quantified. As shown in FIG. 11, the deoxycholate method again showed that it had a lower concentration of DNA than the Triton and Deoxycholate method combined.

### EXAMPLE 7

In order to optimize decellularization conditions. The colon were first incubated in a water bath for 1 hour at 37° C. in a solution of 0.02% Trypsin/0.05% EDTA (10 ml solution per 1 g of ECM). Upon incubation, the colon were washed twice in water for 5 minutes at 300 RMP, and subsequently stored at 4° C. The next day, the colon were washed with 19:1 ml of 4% deoxycholate to 1 g of ECM for 30 minutes at 300 RPM, followed by two washes with water for 5 minutes, the washes were repeated.

DNA was extracted and quantified. As shown in FIG. 12, the 4% deoxycholate method resulted in a remaining DNA content of around 50 ng/mg. 50 ng/mg is the threshold criteria for assessing whether or not the decellularization method is effective. Results here show that ratio of 10 ml of Trypsin/EDTA to 1 g of ECM and the ratio 19 ml 4% Deoxycholate to 1 g of ECM worked effectively.

### EXAMPLE 8

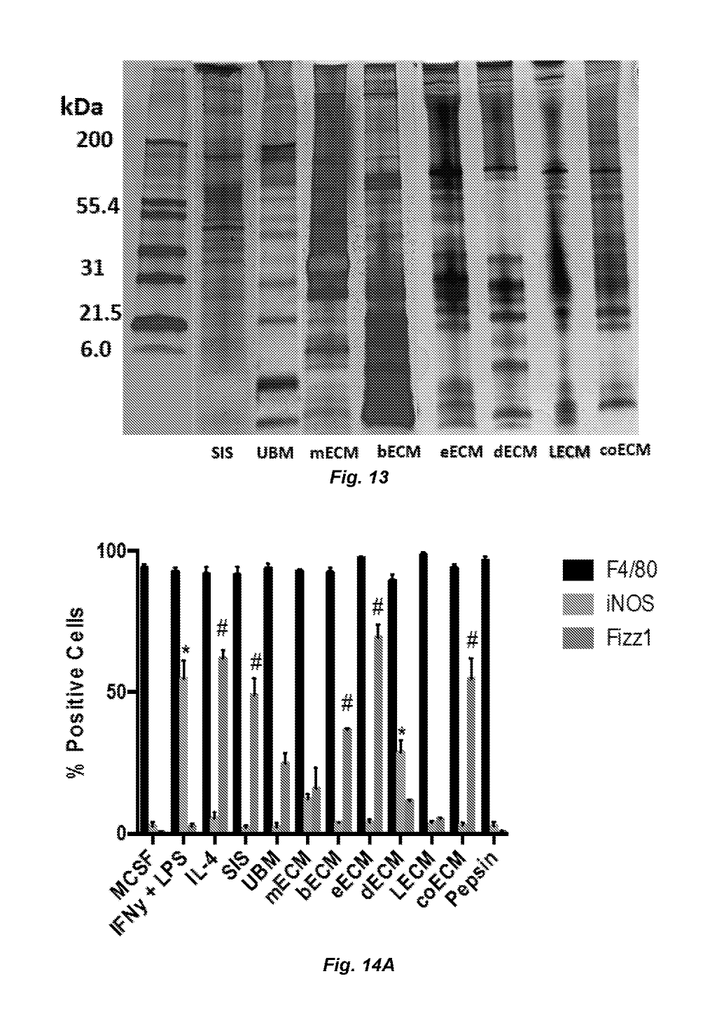

Colon ECM has unique protein content. FIG. 13 shows SDS PAGE gel analysis of ECM degradation products. ECM bioscaffolds were prepared from various different source tissues (SIS=small intestinal submucosa, UBM=urinary bladder matrix, mECM=skeletal muscle ECM, bECM=brain ECM, eECM=esophageal ECM, dECM=dermal ECM, LECM=liver ECM, coECM=colonic ECM) according to established decellularization protocols. All samples were degraded using pepsin. Degradation products of ECM bioscaffolds derived from different source tissues were separated using SDS PAGE gel electrophoresis and show distinct banding patterns.

### EXAMPLE 9

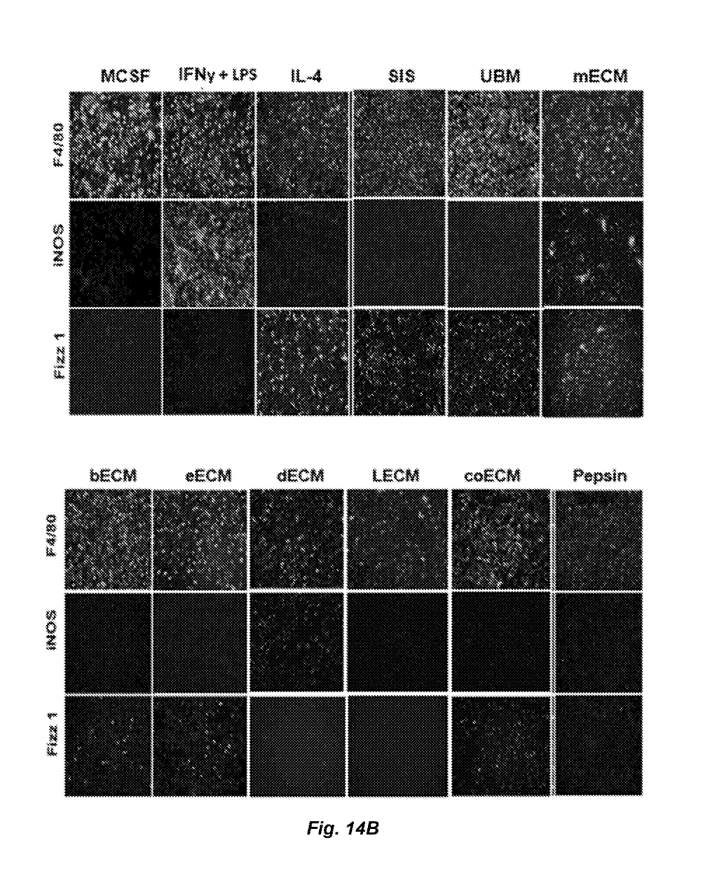

FIG. 14A and 14B illustrate that colon ECM supports macrophage activation towards M2. Immunolabeling of ECM treated macrophages. For FIG. 14B, macrophages were fixed with 2% paraformaldehyde following 18 hours of treatment with cytokines or ECM degradation products and immunolabeled for indicators of the M1 or M2 phenotypes (iNOS, Fizz1, respectively). F4/80 was used as a pan macrophage marker. For FIG. 14A, results were quantified using CellProfiler Image analysis software and show that SIS, bECM, eECM, and coECM promote a predominant M2-like macrophage phenotype, whereas dECM promotes a predominant M1-like macrophage phenotype. (MCSF=macrophage colony stimulating factor, SIS=small intestinal submucosa, UBM=urinary bladder matrix, mECM=skeletal muscle ECM, bECM=brain ECM, eECM=esophageal ECM, dECM=dermal ECM, LECM=liver ECM, coECM=colonic ECM). (* and # indicate p<0.05 when compared to MCSF group for iNOS and Fizz1 quantification, respectively. n=8. Error bars represent standard error of the mean. Light exposure times were standardized to a negative isotype control and kept constant across images, cells were seeded at 2 million cells per well with an average of 900 nuclei counted per field of view).

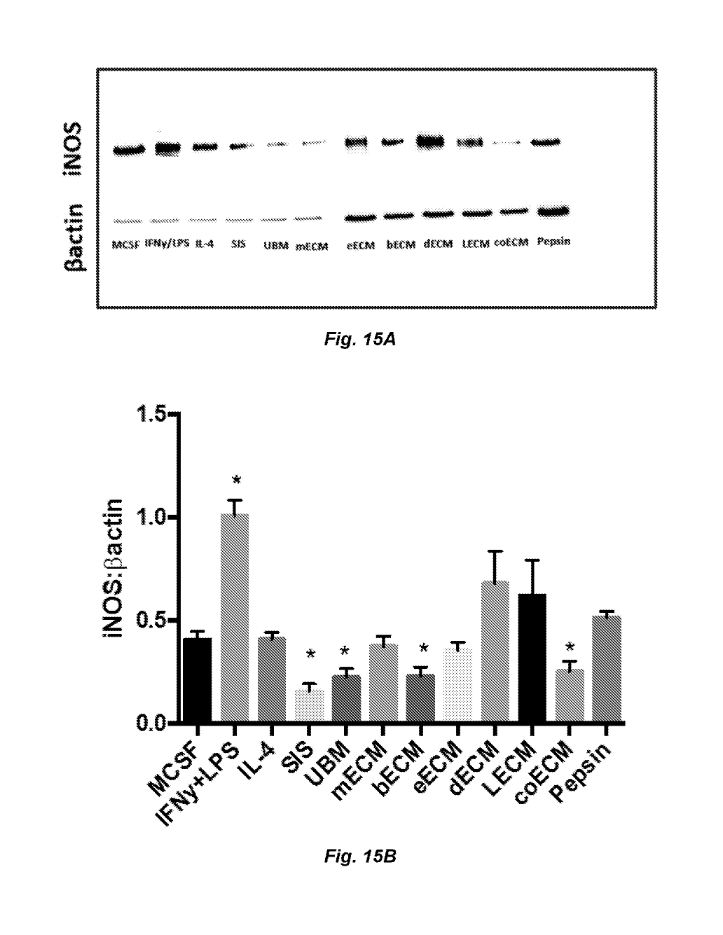

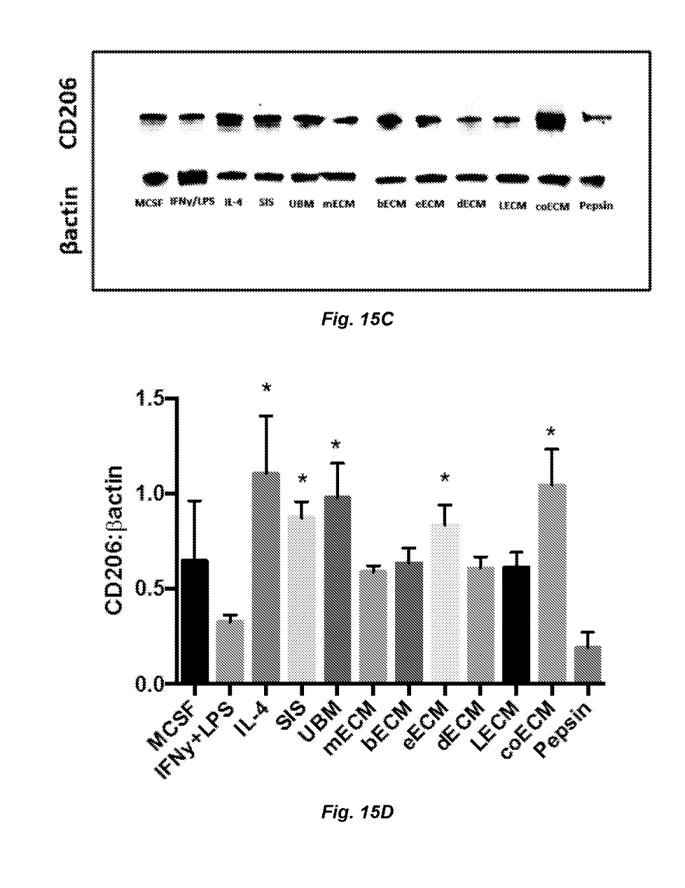

FIG. 15A-15D provide Western blotting of ECM treated macrophages. For FIG. 15A, macrophage lysates were collected and probed for the presence of iNOS and for FIG. 15C, CD206 as M1 and M2-like protein markers, respectively. For FIG. 15B, treatment with SIS, UBM, bECM, and coECM promotes a significant decrease in iNOS expression when compared to the vehicle (pepsin) control treatment. For FIG. 15D, treatment with SIS, UBM, eECM, and coECM promotes an increase in CD206 expression similarly to IL-4 treated macrophages when compared to pepsin treated macrophages. (MCSF=macrophage colony stimulating factor, SIS=small intestinal submucosa, UBM=urinary bladder matrix, mECM=skeletal muscle ECM, bECM=brain ECM, eECM=esophageal ECM, dECM=dermal ECM, LECM=liver ECM, coECM=colonic ECM, * indicates p<0.05 compared to the vehicle control treatment, error bars represent standard error of the mean, n=6).

### EXAMPLE 10

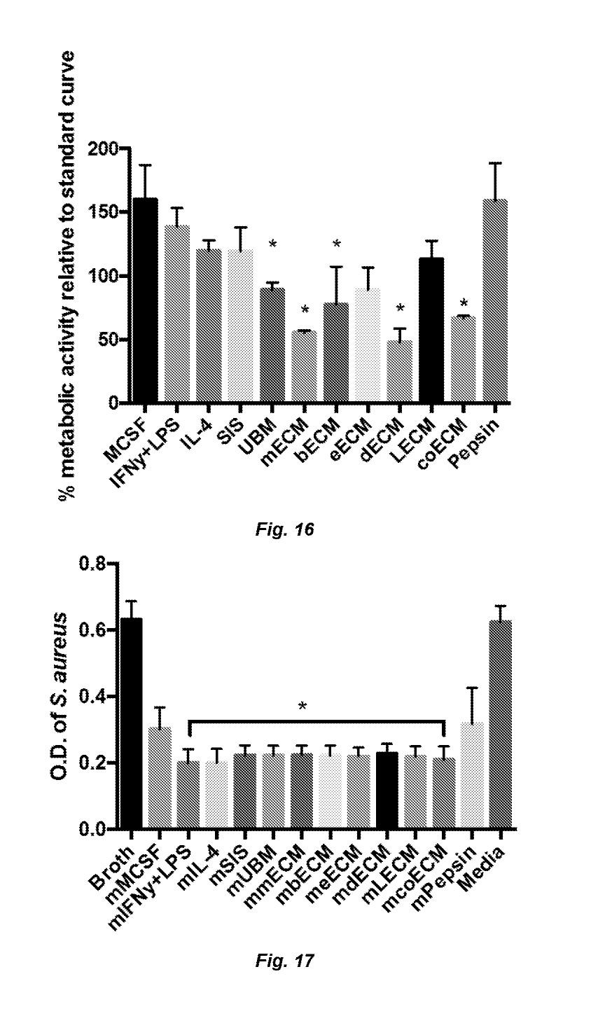

FIG. 16 illustrates that colon ECM decreases metabolic activity of macrophages. MTT analysis was provided using Vybrant® MTT Cell Proliferation Assay Kit (Thermo Fisher Scientific, Pittsburgh Pa.) per manufacturer's guidelines. Results show that treatment with pepsin-digested mECM, dECM, coECM, or bECM reduces metabolic activity of macrophages when compared to the untreated control. UBM, mECM, bECM, dECM, and coECM result in a significant decrease in MTT metabolism when compared to untreated macrophages. (MCSF=macrophage colony stimulating factor, SIS=small intestinal submucosa, UBM=urinary bladder matrix, mECM=skeletal muscle ECM, bECM=brain ECM, eECM=esophageal ECM, dECM=dermal ECM, LECM=liver ECM, coECM=colonic ECM, * indicates p<0.05 when compared to untreated macrophages, error bars represent standard deviation, n=3).

### EXAMPLE 11

Antimicrobial peptides (AMPS), secreted by cells of the innate immune system, represent one of the principal mechanisms of direct defense against bacterial infection binding and neutralizing lipopolysaccharides and protecting the cells against endotoxic shock. The AMP cathelicidin LL-37 is produced by macrophages and actively affects the stability of bacteria (Gram+ and Gram−), viruses, parasites, and fungi, therefore providing protection in a wide range of infections.

Colon ECM degradation products exhibits indirect antimicrobial activity, as shown in FIG. 17. S. aureus growth was used to determine the antimicrobial effects of macrophages exposed to ECM pepsin degradation products. After 18 hours, secreted products from pepsin-digested ECM-treated macrophages significantly inhibit S. aureus growth, similarly to cytokine-treated macrophages, when compared to untreated macrophages and the negative control (broth). (m indicates macrophages treated with the following MCSF=macrophage colony stimulating factor, SIS=small intestinal submucosa, UBM=urinary bladder matrix, mECM=skeletal muscle ECM, bECM=brain ECM, eECM=esophageal ECM, dECM=dermal ECM, LECM=liver ECM, coECM=colonic ECM, * indicates p<0.05 when compared to broth, error bars represent standard deviation, n=4).

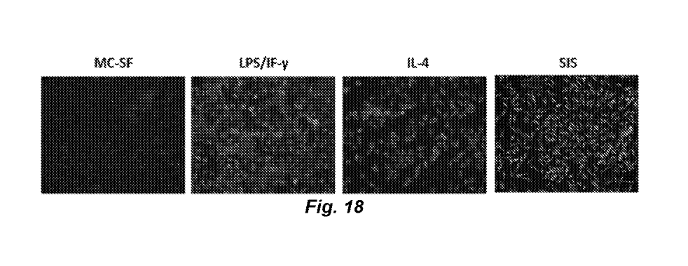

As shown in FIG. 18, stimulation of macrophages with pepsin-digested SIS induces the expression of the antimicrobial peptide cathelicidin LL-37. Representative images of cathelicidin expressing macrophages (green in original) in vitro. Murine bone marrow-derived macrophages were stimulated with pro-inflammatory (LPS/IF-γ), anti-inflammatory (IL-4) cytokines, or extracellular matrix (ECM) digest derived from small intestinal submucosa (SIS) for 24 hours. N-value=5, triplicates). It is expected that colon ECM acts indirectly in the same manner to induce expression of cathelicidin LL-37.

The following clauses provide exemplary aspects of the invention.

Clause 1: A method for producing decellularized colonic extracellular matrix material, comprising, in order:
delipidizing colon tissue with chloroform and a C₁-C₄ alcohol;
digesting the delipidized colon tissue with a protease;
washing the protease-digested colon tissue with deoxycholic acid or a salt thereof, such as sodium deoxycholate; and
disinfecting the washed colon tissue.

Clause 2: The method of clause 1, wherein the colon tissue is delipidized with a mixture of chloroform and methanol followed by one or more washes with ethanol.

Clause 3: The method of clause 1 or 2 wherein the protease is Trypsin.

Clause 4: The method of any of clauses 1-3, wherein the protease is provided as a Trypsin/EDTA composition.

Clause 5: The method of any of clauses 1-4, wherein at least 85%, 90, or 95% of phospholipids are removed.

Clause 6: The method of any of clauses 1-5 wherein at least 95% of the DNA of the colon tissue is removed.

Clause 7: The method of any of clauses 1-6, wherein any residual DNA in the colon tissue is in fragments of <200 bases in length. Clause 8: The method of any of clauses 1-7, wherein hyaluronic acid is digested less than 50%, 40%, 30%, 25%, 20% or 10%.

Clause 9: The method of any of clauses 1-5, wherein sulfated glycosaminoglycans are digested less than 50%, 40%, 30%, 25%, 20% or 10%.

Clause 10: The method of any of clauses 1-9, wherein the amount of collagen in the colon tissue is enriched by at least 3 fold.

Clause 11: The method of any of clauses 1-10, further comprising, after disinfecting the colon tissue:
digesting the tissue in an acid protease; and
raising the pH of the acid-protease-digested tissue to a pH ranging from 7.2 to 7.8, from 7.3 to 7.5, or 7.4 to produce a pre-gel.

Clause 12: The method of clause 11, further comprising, after disinfecting the colon tissue and prior to digesting the tissue in an acid protease, comminuting the colon tissue.

Clause 13: The method of either of clauses 11 or 12, further comprising during or after digesting the tissue in an acid protease cooling the sample to from 0° C. to below 25° C., 20° C., 15° C., 10° C., or 5° C., such as to 4° C., and, after digesting the tissue in the acid protease, optionally raising the pH of the acid-protease-digested tissue to a pH ranging from 7.2 to 7.8, e.g., 7.3 to 7.5, or 7.4 to produce a pre-gel.

Clause 14: The method of any of clauses 11-13, further comprising warming the pre-gel to a temperature at which the pre-gel forms a hydrogel.

Clause 15: The method of any of clauses 1-14, wherein the colon tissue is isolated colon submucosa.

Clause 16: The method of any of clauses 1-10, wherein the colon tissue is porcine.

Clause 17: The method of any of clauses 1-16, further comprising lyophilizing the decellularized colonic extracellular matrix material.

Clause 18: The method of any of clauses 1-17, comprising a washing step between any of the steps.

Clause 19: A method of treating a defective, diseased, or damaged tissue or organ in a patient comprising implanting, injecting or otherwise introducing the decellularized colonic extracellular matrix material of any of clauses 1-18 into, on, or about the defective, diseased, or damaged tissue or organ in the patient.

Clause 20: The method of claim 19, resulting in a increased decellularized colonic extracellular matrix material in the patient.

Clause 21: The method of either of clauses 19 or 20. wherein the decellularized colonic extracellular matrix material is degraded in the patient in less than 50, or less than 40 days.

Clause 22: A method of treating esophageal disease, a method of treating short bowel syndrome, a method of treating ulcerative colitis, a method of treating Crohn's disease, or a method of treating mucositis in a patient, comprising implanting, injecting or otherwise introducing the decellularized colonic extracellular matrix material of any of clauses 1-18 into, on or about defective, diseased, or damaged tissue or organ of the patient.

Clause 23: A decellularized colonic extracellular matrix material prepared according to any one of clauses 1-18.

Clause 24: Use of a decellularized colonic extracellular matrix material prepared according to any one of clauses 1-18, for treatment of a defective, diseased, or damaged tissue or organ in a patient.

Clause 25: Use of a decellularized colonic extracellular matrix material prepared according to any one of clauses 1-18, for treating esophageal disease, for treating short bowel syndrome, for treating ulcerative colitis, for treating Crohn's disease, or for treating mucositis in a patient.

Having described this invention, it will be understood to those of ordinary skill in the art that the same can be performed within a wide and equivalent range of conditions, formulations and other parameters without affecting the scope of the invention or any embodiment thereof.

1. A method for producing decellularized colonic extracellular matrix material, comprising:
a. delipidizing colon tissue with chloroform and a C₁-C₄ alcohol;
b. digesting the delipidized colon tissue with a protease;
c. washing the protease-digested colon tissue with deoxycholic acid or a salt thereof, such as sodium deoxycholate; and
d. disinfecting the washed colon tissue.

2. The method of claim 1, wherein the colon tissue is delipidized with a mixture of chloroform and methanol followed by one or more washes with ethanol.

3. The method of claim 1, wherein the protease is Trypsin.

4. The method of claim 1, wherein the protease is provided as a Trypsin/EDTA composition.

5. The method of claim 1, wherein at least 85% of phospholipids are removed.

6. The method of claim 1 wherein at least 95% of the DNA of the colon tissue is removed.

7. The method of claim 1, wherein any residual DNA in the colon tissue is in fragments of <200 bases in length.

8. The method of claim 1, wherein hyaluronic acid is digested less than 50%.

9. The method of claim 1, wherein sulfated glycosaminoglycans are digested less than 50%.

10. The method of claim 1, wherein the amount of collagen in the colon tissue is enriched by at least 3 fold.

11. The method of claim 1, further comprising, after disinfecting the colon tissue:
a. digesting the tissue in an acid protease; and
b. raising the pH of the acid-protease-digested tissue to a pH ranging from 7.2 to 7.8 to produce a pre-gel.

12. The method of claim 11, further comprising, after disinfecting the colon tissue and prior to digesting the tissue in an acid protease, comminuting the colon tissue.

13. The method of claim 11, further comprising during or after digesting the tissue in an acid protease cooling the sample to from 0° C. to below 25° C. and, after digesting the tissue in the acid protease, optionally raising the pH of the acid-protease-digested tissue to a pH ranging from 7.2 to 7.8 to produce a pre-gel.

14. The method of claim 11, further comprising warming the pre-gel to a temperature at which the pre-gel forms a hydrogel.

15. The method of claim 1, wherein the colon tissue is isolated colon submucosa.

16. The method of claim 1, further comprising lyophilizing the decellularized colonic extracellular matrix material.

17. The method of claim 1, comprising a washing step between any of the steps.

18. A decellularized colonic extracellular matrix material prepared according to claim 1.

19. (canceled)

20. (canceled)

21. A method of treating a defective, diseased, or damaged tissue or organ in a patient comprising introducing an effective amount of the decellularized colonic extracellular matrix material of claim 1 into, on, or about the defective, diseased, or damaged tissue or organ in the patient.

22. The method of claim 21, wherein the defective diseased damaged tissue or organ in the patient is associated with esophageal disease, short bowel syndrome, ulcerative colitis, Crohn's disease, or mucositis in a patient.
